(19) United States
(12) Patent Application Publication
(10) Pub. No.: US 2025/0339323 A1
(43) Pub. Date: Nov. 6, 2025

(54) PRESSURE-MITIGATION APPARATUSES DESIGNED TO BE FASTENED TO, OR INTEGRATED INTO, SUPPORTIVE SUBSTRATES AND APPROACHES TO USING THE SAME TO ALLEVIATE PRESSURE

(71) Applicant: TurnCare, Inc., Palo Alto, CA (US)

(72) Inventor: Rafael Paolo Squitieri, Wilton, CT (US)

(21) Appl. No.: 19/268,898

(22) Filed: Jul. 14, 2025

Related U.S. Application Data
(63) Continuation of application No. PCT/US2024/011490, filed on Jan. 12, 2024.
(60) Provisional application No. 63/480,271, filed on Jan. 17, 2023.

(51) Int. Cl. A61G 7/057

(52) U.S. Cl. CPC A61G 7/05769

(57) ABSTRACT

Introduced here are pressure-mitigation devices having improved stabilization and reduced slippage when in use. Pressure-mitigation devices can be configured to directly fit and/or fasten to substrates that support whole human bodies or parts of human bodies. As an example, a pressure-mitigation device can be incorporated into a fitted mattress cover that fits a mattress. Other example embodiments include pressure-mitigation mattress sheets, cushion sleeves, pillow covers, and/or the like. According to some embodiments, pressure-mitigation devices are directly incorporated into a substrate. As an example, a pressure-mitigation device can be directly incorporated as an upper layer of a cushion or mattress. Together, the pressure-mitigation upper layer and other cushioning layers (e.g., transition layers, coil or spring bases, comfort layers, support layers) can form an integrated and unitary pressure-mitigation substrate. The pressure-mitigation substrate can be directly used as (or wholly replace) a seat cushion, a wheelchair cushion, a bed mattress, and/or the like.

### CROSS-REFERENCE TO RELATED APPLICATIONS

This application is a continuation of International Application No. PCT/US2024/011490, titled “Pressure-Mitigation Apparatuses Designed to be Fastened to, or Integrated into, Supportive Substrates and Approaches to Using the Same to Alleviate Pressure” and filed Jan. 12, 2024, which claims priority to U.S. Provisional Application No. 63/480,271, titled “Pressure-Mitigation Apparatuses Designed to be Fastened to, or Integrated into, Supportive Substrates and Approaches to Using the Same to Alleviate Pressure” and filed Jan. 17, 2023, which are incorporated herein by reference in their entireties.

### TECHNICAL FIELD

Various embodiments concern pressure-mitigation apparatuses able to apply force to two or more anatomical areas of a human by two or more objects.

### BACKGROUND

Pressure injuries—sometimes referred to as “decubitus ulcers,” “pressure ulcers,” “pressure sores,” or “bedsores”—may occur as a result of steady pressure being applied in one location along the surface of the human body for a prolonged period of time. Regions with bony prominences are especially susceptible to pressure injuries. Pressure injuries are most common in individuals who are completely immobilized (e.g., on an operating table, bed, or chair) or have impaired mobility. These individuals may be older, malnourished, or incontinent, all factors that predispose the human body to formation of pressure injuries.

These individuals are often not ambulatory, so they sit or lie for prolonged periods of time in the same position. Moreover, these individuals may be unable to reposition themselves to alleviate pressure. Consequently, pressure on the skin and underlying soft tissue may eventually result in inadequate blood flow to the area, a condition referred to as “ischemia,” thereby resulting in damage to the skin or underlying soft tissue. Pressure injuries can take the form of a superficial injury to the skin or a deeper ulcer that exposes the underlying tissues and places the individual at risk for infection. The resulting infection may worsen, leading to sepsis or even death in some cases.

There are various technologies on the market that profess to prevent pressure injuries. However, these conventional technologies have many deficiencies. For instance, these conventional technologies are unable to control the spatial relationship between a human body and a support surface (or simply “surface”) that applies pressure to the human body. Conventional technologies are also unable to effectively coordinate the use of multiple surfaces that apply pressure to various parts of the human body. Consequently, individuals that use these conventional technologies have to operate multiple devices that control multiple surfaces, with the outcome being that they may still develop pressure injuries or suffer from related complications.

### BRIEF DESCRIPTION OF THE DRAWINGS

FIGS. 1A-B are top and bottom views, respectively, of a pressure-mitigation device able to relieve the pressure on an anatomical region applied by the surface of an elongated object in accordance with embodiments of the present technology.

FIGS. 2A and 2B are top and bottom views, respectively, of a pressure-mitigation device configured in accordance with embodiments of the present technology.

FIG. 3 is a top view of a pressure-mitigation device for relieving pressure on an anatomical region applied by a wheelchair in accordance with embodiments of the present technology.

FIGS. 4A and 4B are perspective views of a pressure-mitigation device that is directly incorporated into a fitted cover for a substrate, for example a mattress, in accordance with embodiments of the present technology.

FIG. 4C is a partially schematic side view of a pressure-mitigation device that is configured to fit and/or fasten to a substrate, for example a mattress, in accordance with embodiments of the present technology.

FIG. 4D is a partially schematic side view of a pressure-mitigation device that is configured to fasten to a substrate, for example a mattress, in accordance with embodiments of the present technology.

FIG. 5 is a flow diagram of a process for deploying a pressure-mitigation device that is configured to fit and/or fasten to a substrate to securely prevent and/or address ischemia-reperfusion injuries in accordance with embodiments of the present technology.

FIG. 6 is a partially schematic top view of a pressure-mitigation device illustrating how a pressure gradient can be created by varying pressure distributions to avoid ischemia in a mobility-impaired patient in accordance with embodiments of the present technology.

FIG. 7A is a partially schematic side view of a pressure-mitigation device for relieving pressure on a specific anatomical region by deflating one or more chambers in accordance with embodiments of the present technology.

FIG. 7B is a partially schematic side view of a pressure-mitigation device for relieving pressure on a specific anatomical region by inflating one or more chambers in accordance with embodiments of the present technology.

FIG. 8A is a perspective view of a pressure-mitigation device directly incorporated into an upper layer of a substrate, such as a mattress, in accordance with embodiments of the present technology.

FIGS. 8B and 8C are partially schematic side views of pressure-mitigation devices directly incorporated into an upper layer of a substrate, such as a mattress, in accordance with embodiments of the present technology.

FIGS. 9A-9C are isometric, front, and back views, respectively, of a controller device (also referred to as a “controller”) that is responsible for controlling inflation and/or deflation of the chambers of a pressure-mitigation device in accordance with embodiments of the present technology.

FIG. 10 illustrates an example of a controller in accordance with embodiments of the present technology.

FIG. 11 is a block diagram illustrating an example of a processing system in which at least some operations described herein can be implemented.

Various features of the technologies described herein will become more apparent to those skilled in the art from a study of the Detailed Description in conjunction with the drawings. Embodiments are illustrated by way of example and not limitation in the drawings. While the drawings depict various embodiments for the purpose of illustration, those skilled in the art will recognize that alternative embodiments may be employed without departing from the principles of the technologies. Accordingly, while specific embodiments are shown in the drawings, the technology is amenable to various modifications.

### DETAILED DESCRIPTION

The term “pressure injury” refers to a localized region of damage to the skin and/or underlying tissue that results from contact pressure (or simply “pressure”) on the corresponding anatomical region of the human body. Pressure injuries will often form over bony prominences, such as the skin and soft tissue overlying the sacrum, coccyx, heels, or hips. However, other sites may also be affected. For instance, pressure injuries may form on the elbows, knees, ankles, shoulders, abdomen, back, or cranium. Pressure injuries may develop when pressure is applied to the blood vessels in soft tissue in such a manner that blood flow to the soft tissue is at least partially obstructed (e.g., due to the pressure exceeding the capillary filling pressure), and ischemia results at the site when such obstruction occurs for an extended duration. Accordingly, pressure injuries are normally observed on individuals who are mobility impaired, immobilized, or sedentary for prolonged periods of times.

Once pressure injuries have formed, the healing process is normally slow. When pressure is relieved from the site of a pressure injury, the body will rush blood (with proinflammatory mediators) to that region to perfuse the area with blood. The sudden reperfusion of the damaged (and previously ischemic) region has been shown to cause an inflammatory response, brought on by the proinflammatory mediators, that can actually worsen the pressure injury (and thus prolong recovery). Moreover, in some cases, the proinflammatory mediators may spread through the blood stream beyond the site of the pressure injury to cause a systematic inflammatory response (also referred to as a “secondary inflammatory response”). Secondary inflammatory responses caused by proinflammatory mediators have been shown to exacerbate existing conditions and/or trigger new conditions, thereby slowing recovery. Recovery can also be prolonged by factors that are frequently associated with individuals who are prone to pressure injuries, such as old age, immobility, preexisting medical conditions (e.g., arteriosclerosis, diabetes, or infection), smoking, and medications (e.g., anti-inflammatory drugs). Inhibiting the formation of pressure injuries (and reducing the prevalence of proinflammatory mediators) can enhance and expedite many treatment processes, especially for those individuals whose mobility is impaired during treatment.

Introduced here, therefore, are pressure-mitigation devices able to mitigate the pressure applied to a human body by the surface of an object (also referred to as a “structure”). A controller device (or simply “controller”) can be fluidically coupled to a pressure-mitigation device (also referred to as a “pressure-mitigation apparatus” or a “pressure-mitigation pad”) that includes a series of selectively inflatable chambers (also referred to as “cells” or “compartments”). When a pressure-mitigation device is placed between a human body and a surface, the controller can continuously, intelligently, and autonomously circulate fluid through the chambers of the pressure-mitigation device. Normally, the controller circulates air through the chambers of the pressure-mitigation device, though the controller could circulate another fluid, such as water or gel, through the chambers of the pressure-mitigation device. As further discussed below, the controller may cause the chambers to be selectively inflated, deflated, or any combination thereof.

The present disclosure is directed to pressure-mitigation devices and apparatuses having improved stabilization and reduced slippage when in use. Example pressure-mitigation devices are configured to directly fit and/or fasten to substrates (e.g., mattresses, pads, cushions, pillows) that support whole human bodies or parts of human bodies. As one example, a pressure-mitigation device can be incorporated into a fitted mattress cover that fits a mattress. That is, a pressure-mitigation mattress cover can include inflatable chambers that spread pressure exerted on a human body atop a mattress, and the pressure-mitigation mattress cover can be configured to wrap around and secure to the mattress. Other example embodiments include pressure-mitigation mattress sheets, cushion sleeves, pillow covers, and/or the like. According to some embodiments, example pressure-mitigation devices are directly incorporated into a substrate. As one illustrative non-limiting example, a pressure-mitigation device can be directly incorporated as an upper layer of a cushion or mattress. Together, the pressure-mitigation upper layer and other cushioning layers (e.g., transition layers, coil or spring bases, comfort layers, support layers) can form an integrated and unitary pressure-mitigation substrate. The pressure-mitigation substrate can be directly used as (or wholly replace) a seat cushion, a wheelchair cushion, a bed mattress, and/or the like.

Example pressure-mitigation apparatuses disclosed herein provide various technical benefits. Effective pressure-mitigation treatment provided by a pressure-mitigation devices is provided primarily while the pressure-mitigation device is disposed between the patient and the substrate on which the patient is disposed. With existing pressure-mitigation devices, movement of the patient (whether actuated by the patient themselves or resulting from the pressure-mitigation treatment of the pressure-mitigation device) can lead to movement, slippage, and displacement of the pressure-mitigation device, and time and resources are expended to repeatedly reposition the pressure-mitigation device and the patient atop the pressure-mitigation device.

Some existing pressure-mitigation devices are configured to be simply placed atop a substrate and rely upon tacky or non-slip material that reduces slippage with the substrate. However, effectiveness of the tacky or non-slip material can be reduced, for example, in the presence of various liquids. Other existing pressure-mitigation devices interface with attachment apparatuses, such as those disclosed in U.S. application Ser. No. 16/363,094, titled “INFLATABLE PERFUSION ENHANCEMENT APPARATUSES AND ASSOCIATED DEVICES, SYSTEMS AND METHODS” and filed on Mar. 25, 2019, and U.S. application Ser. No. 17/495,072, titled “INFLATABLE PERFUSION ENHANCEMENT APPARATUSES AND ASSOCIATED DEVICES, SYSTEMS AND METHOD” and filed on Oct. 6, 2021, the contents of each of these identified applications being incorporated by referenced herein in their respective entireties. These attachment apparatuses can act as a separate link between a pressure-mitigation device and a substrate, resulting in an increase of total equipment required for pressure-mitigation treatment. Embodiments disclosed herein provide technical improvements by securely and directly integrating a pressure-mitigation device with a substrate, whether via a cover, sleeve, or the like that fits a substrate or into the substrate itself.

Those skilled in the art will recognize that a pressure-mitigation device can “fit” a substrate in various ways. For example, some pressure-mitigation devices may be designed and/or installed to occupy an entire surface of a substrate, such as the entire surface of a mattress, while other pressure-mitigation devices may be designed and/or installed to occupy a portion of the surface of a substrate, such as only the central section defined longitudinally along the length of a mattress. Additionally or alternatively, pressure-mitigation devices could include fastening mechanisms (also called “securement mechanisms”) that allow the pressure-mitigation devices to be readily secured to, yet remain detachable from, a substrate. Consider, for example, a pressure-mitigation device that is designed to occupy the entire surface of a mattress. In such a scenario, the pressure-mitigation device may have fastening mechanisms (e.g., elastic straps, clips, magnets) for facilitating securement to the periphery of the mattress. The nature, number, and location of fastening mechanisms may vary depending on the nature of the substrate (and therefore, the nature of the pressure-mitigation device).

Embodiments may be described with reference to particular anatomical regions, treatment regimens, environments, etc. However, those skilled in the art will recognize that the features are similarly applicable to other anatomical regions, treatment regimens, environments, etc. As an example, embodiments may be described in the context of a pressure-mitigation device that is positioned adjacent to an anterior anatomical region of an individual oriented in the prone position. However, aspects of those embodiments may apply to a pressure-mitigation device that is positioned adjacent to a posterior anatomical region of an individual oriented in the supine position.

While embodiments may be described in the context of machine-readable instructions, aspects of the technology can be implemented via hardware, firmware, or software. As an example, a controller may not only execute instructions for determining an appropriate rate at which to permit fluid (e.g., air) flow into the inflatable chamber of a pressure-mitigation device but may also be responsible for facilitating communication with other computing devices. For example, the controller may be able to communicate with a mobile device that is associated with the individual or a caregiver, or the controller may be able to communicate with a computer server of a network-accessible server system.

### Terminology

References in this description to “an embodiment” or “one embodiment” means that the feature, function, structure, or characteristic being described is included in at least one embodiment of the technology. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” and variants thereof are intended to include any connection or coupling between two or more elements, either direct or indirect. The connection/coupling can be physical, logical, or a combination thereof. For example, objects may be electrically or communicatively coupled to one another despite not sharing a physical connection.

The term “module” may refer to software components, firmware components, or hardware components. Modules are typically functional components that generate one or more outputs based on one or more inputs. As an example, a computer program may include multiple modules responsible for completing different tasks or a single module responsible for completing all tasks.

When used in reference to a list of multiple items, the term “or” is intended to cover all of the following interpretations: any of the items in the list, all of the items in the list, and any combination of items in the list.

The sequences of steps performed in any of the processes described here are exemplary. However, unless contrary to physical possibility, the steps may be performed in various sequences and combinations. For example, steps could be added to, or removed from, the processes described here. Similarly, steps could be replaced or reordered. Thus, descriptions of any processes are intended to be open ended.

### Overview of Pressure-Mitigation Devices

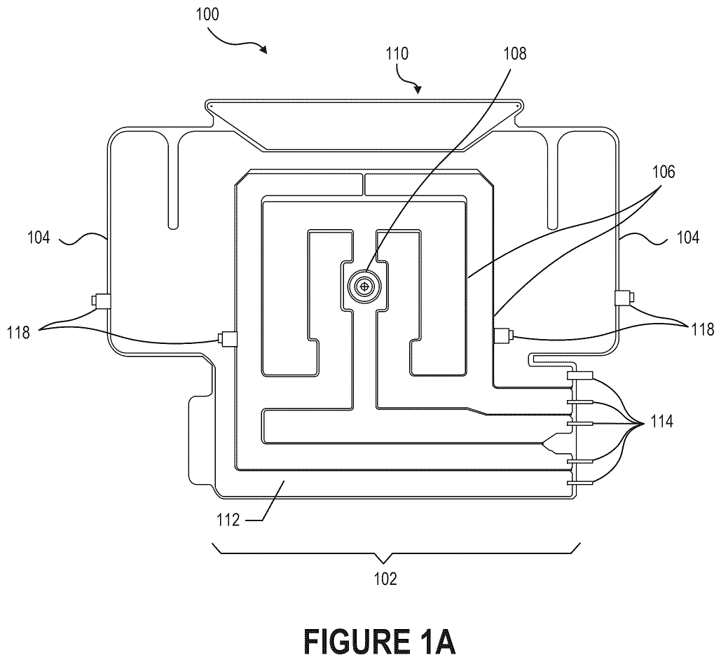

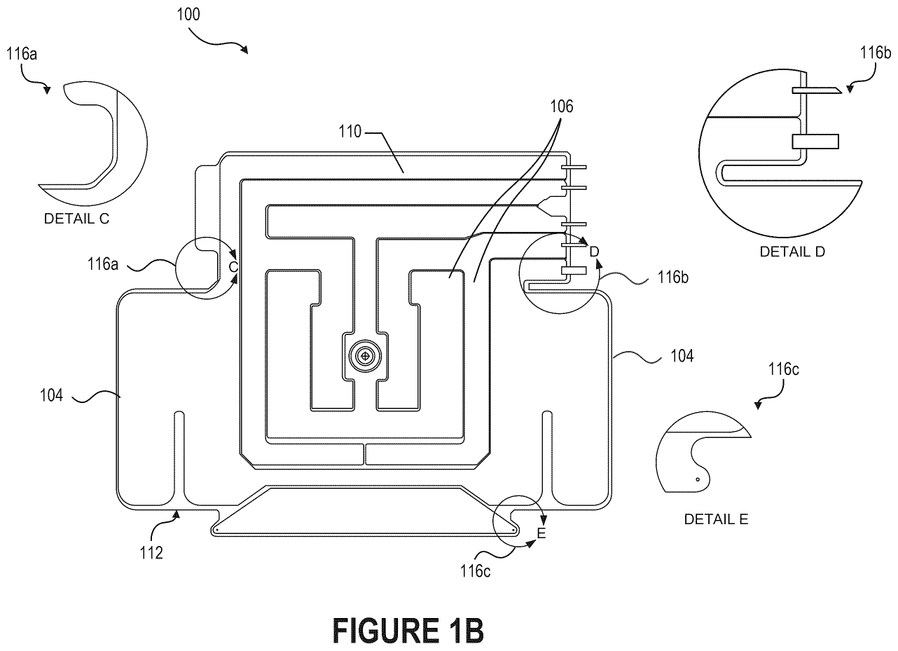

A pressure-mitigation apparatus includes a plurality of chambers or compartments that can be individually controlled to vary the pressure in each chamber and/or a subset of the chambers. When placed between a human body and a support surface, the pressure-mitigation apparatus can vary the pressure on an anatomical region by controllably inflating one or more chambers, deflating one or more chambers, or any combination thereof. Several examples of pressure-mitigation apparatuses are described below with respect to FIGS. 1A-3. Unless otherwise noted, any features described with respect to one embodiment are equally applicable to the other embodiments. Some features have only been described with respect to a single embodiment of the pressure-mitigation apparatus for the purpose of simplifying the present disclosure.

FIGS. 1A-B are top and bottom views, respectively, of an example of a pressure-mitigation device 100, able to relieve the pressure on an anatomical region applied by the surface of an elongated object in accordance with embodiments of the present technology. While the pressure-mitigation device 100 may be described in the context of elongated objects, such as mattresses, stretchers, operating tables, and procedure tables, the pressure-mitigation device 100 could be deployed on non-elongated objects.

In some embodiments, the pressure-mitigation device 100 is secured to a support surface or substrate (e.g., a mattress, a cushion, a pad) using an attachment apparatus. In other embodiments, the pressure-mitigation device 100 is placed in direct contact with the surface without any attachment apparatus therebetween. For example, the pressure-mitigation device 100 may have a tacky substance deposited along at least a portion of its outer surface that allows it to temporarily adhere to the surface. Examples of tacky substances include latex, urethane, and silicone rubber. However, as discussed, these techniques involve additional equipment or materials, and reliability of such techniques are improved upon in embodiments disclosed herein.

As shown in FIG. 1A, the pressure-mitigation device 100 can include a central portion 102 (also referred to as a “contact portion”) that is positioned alongside at least one side support 104. Here, a pair of side supports 104 are arranged on opposing sides of the central portion 102. However, some embodiments of the pressure-mitigation device 100 do not include any side supports. For example, the side support(s) 104 may be omitted when the individual is medically immobilized (e.g., under anesthesia, in a medically induced coma, etc.) and/or physically restrained by underlying object (e.g., by rails along the side of a bed, armrests along the side of a chair, etc.) or some other structure (e.g., physical restraints, casts, etc.).

The pressure-mitigation device 100 includes a series of chambers 106 whose pressure can be individually varied. In some embodiments, the series of chambers 106 are arranged in a geometric pattern designed to relieve pressure on specific anatomical region(s) of a human body. As noted above, when placed between the human body and a surface, the pressure-mitigation device 100 can vary the pressure on these specific anatomical region(s) by controllably inflating and/or deflating chamber(s).

In some embodiments, the series of chambers 106 are arranged such that pressure on a given anatomical region is mitigated when the given anatomical region is oriented over a target region 108 of the geometric pattern. As shown in FIGS. 1A-B, the target region 108 may be representative of a central point of the pressure-mitigation device 100 to appropriately position the anatomy of the human body with respect to the pressure-mitigation device 100. For example, the target region 108 may correspond to an epicenter of the geometric pattern. However, the target region 108 may not necessarily be the central point of the pressure-mitigation device 100, particularly if the series of chambers 106 are positioned in a non-symmetric arrangement. The target region 108 may be visibly marked so that an individual can readily align the target region 108 with a corresponding anatomical region of the human body to be positioned thereon. Thus, the pressure-mitigation device 100 may include a visual element representative of the target region 108 to facilitate alignment with the corresponding anatomical region of the human body. The individual could be a physician, nurse, caregiver, or the patient.

The pressure-mitigation device 100 can include a first portion 110 (also referred to as a “first layer” or “bottom layer”) designed to face a surface and a second portion 112 (also referred to as a “second layer” or “top layer”) designed to face the human body supported by the surface. In some embodiments, the pressure-mitigation device 100 is deployed such that the first portion 110 is directly adjacent to the surface. For example, the first portion 110 may have a tacky substance deposited along at least a portion of its exterior surface that facilitates temporarily adhesion to the support surface. In other embodiments, the pressure-mitigation device 100 is deployed such that the first portion 110 is directly adjacent to an attachment apparatus designed to help secure the pressure-mitigation device 100 to the support surface. The pressure-mitigation device 100 may be constructed of various materials, and the material(s) used in the construction of each component of the pressure-mitigation device 100 may be chosen based on the nature of the body contact, if any, to be experienced by the component. For example, because the second portion 112 will often be in direct contact with the skin, it may be comprised of a soft fabric or a breathable fabric (e.g., comprised of moisture-wicking materials or quick-drying materials, or having perforations). In some embodiments, an impervious lining (e.g., comprised of polyurethane) is secured to the inside of the second portion 112 to inhibit fluid (e.g., sweat) from entering the series of chambers 106. As another example, if the pressure-mitigation device 100 is designed for deployment beneath a cover (e.g., a bed sheet), then the second portion 112 may be comprised of a flexible, liquid-impervious material, such as polyurethane, polypropylene, silicone, or rubber. The first portion 110 may also be comprised of a flexible, liquid-impervious material.

Generally, the first and second portions 110, 112 are selected and/or designed such that the pressure-mitigation device 100 is readily cleanable. However, the specific materials that are used may vary depending on the environment in which the pressure-mitigation device 100 is to be deployed. Assume, for example, that the pressure-mitigation device 100 is intended to be deployed in a hospital environment. In such a scenario, the first and second portions 110, 112 may be readily cleanable with a cleaning agent (e.g., bleach) or a cleaning procedure (e.g., sterilization). Because the pressure-mitigation device 100 will remain in the hospital environment under the care of knowledgeable persons, the first and second portions 110, 112 could be comprised of materials that may degrade quickly if not properly cared for. Examples of such materials include high-performance fabric, upholstery, vinyl, and other suitable textiles. If the pressure-mitigation device 100 is instead intended to be deployed in a home environment, the first and second portions 110, 112 may be comprised of materials that can be readily cleaned by persons without extensive experience. For example, the first portion 110 and/or the second portion 112 may be comprised of a vinyl that is easy to clean with commonly available cleaning agents (e.g., bleach, liquid dish soap, all-purpose cleaners). Regardless of the environment, the first and second portions 110, 112 may contain antimicrobial additives, antifungal additives, flame-retardant additives, and the like.

The series of chambers 106 may be formed via interconnections between the first and second portions 110, 112. For example, the first and second portions 110, 112 may be bound directly to one another, or the first and second portions 110, 112 may be bound to one another via one or more intermediary layers. In the embodiment illustrated in FIGS. 1A-B, the pressure-mitigation device 100 includes an “M-shaped” chamber intertwined with two “C-shaped” chambers that face one another. Such an arrangement has been shown to effectively mitigate the pressure applied to the sacral region of a human body in the supine position by a support surface when the pressure in these chambers is alternated. The series of chambers 106 may be arranged differently if the pressure-mitigation device 100 is designed for an anatomical region other than the sacral region, or if the pressure-mitigation device 100 is to be used to support a human body in a non-supine position (e.g., a prone position or sitting position). Generally, the geometric pattern of chambers 106 is designed based on the internal anatomy (e.g., the muscles, bones, and vasculature) of the anatomical region on which pressure is to be relieved.

The person using the pressure-mitigation device 100 and/or the caregiver (e.g., a nurse, physician, family member, etc.) may be responsible for actively orienting the anatomical region of the human body lengthwise over the target region 108 of the geometric pattern. If the pressure-mitigation device 100 includes one or more side supports 104, the side support(s) 104 may actively orient or guide the anatomical region of the human body laterally over the target region 108 of the geometric pattern. In some embodiments the side support(s) 104 are inflatable, while in other embodiments the side support(s) 104 are permanent structures that protrude from one or both lateral sides of the pressure-mitigation device 100. For example, at least a portion of each side support may be stuffed with cotton, latex, polyurethane foam, or any combination thereof.

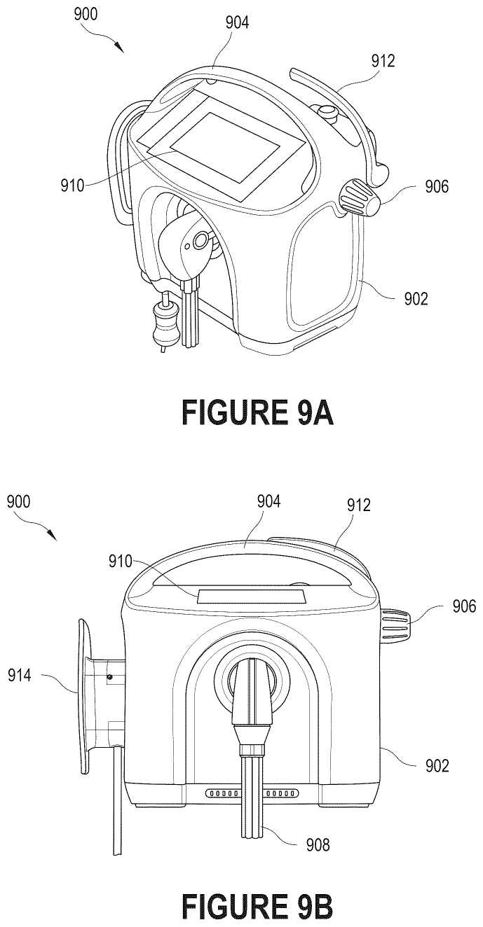

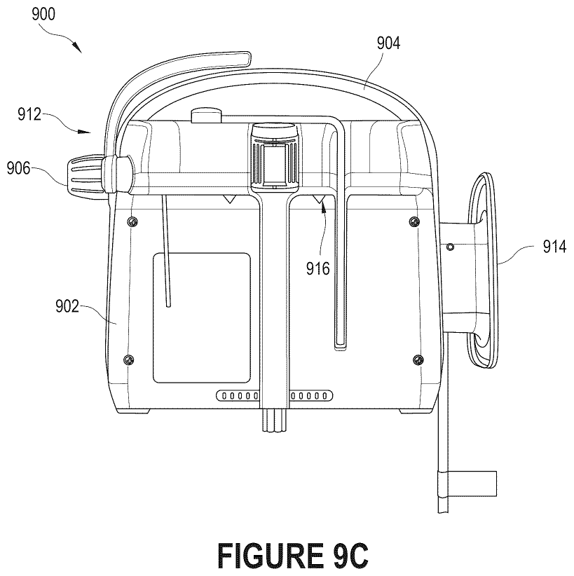

As further described below (e.g., with respect to FIGS. 9A-C), a controller can separately or independently control the pressure in each chamber (as well as the side supports 104, if included) by providing a discrete airflow via one or more corresponding valves 114. In some embodiments, the valves 114 are permanently secured to the pressure-mitigation device 100 and designed to interface with tubing that can be readily detached (e.g., for easier transport, storage, etc.). Here, the pressure-mitigation device 100 includes five valves 114. Three valves are fluidically coupled to the series of chambers 106, and two valves are fluidically coupled to the side supports 104. Other embodiments of the pressure-mitigation device 100 may include more than five valves or less than five valves. For example, the pressure-mitigation device 100 may be designed such that a pair of side supports 104 are pressurized via a single airflow received via a single valve.

In some embodiments, the pressure-mitigation device 100 includes one or more design features 116a-c designed to facilitate securement of the pressure-mitigation device 100 to the surface of an object and/or an attachment apparatus. As illustrated in FIG. 1B, for example, the pressure-mitigation device 100 may include three design features 116a-c, each of which can be aligned with a corresponding structural feature that is accessible along the surface of the object or the attachment apparatus. For example, each design feature 116a-c may be designed to at least partially envelope a structural feature that protrudes upward. One example of such a structural feature is a rail that extends along the side of a bed. The design feature(s) 116a-c may also facilitate proper alignment of the pressure-mitigation device 100 with the surface of the object or the attachment apparatus.

While not shown in FIGS. 1A-B, one or more release valves (also referred to as “discharge valves”) may be located along the periphery of the pressure-mitigation device 100 to allow for quick discharge of the fluid stored therein. Normally, the release valve(s) are located along the longitudinal sides to ensure that the release valve(s) are not located beneath a human body situated on the pressure-mitigation device 100. Release valve(s) may allow discharge of fluid from the side supports 104 and/or the series of chambers 106. In some embodiments, fluid is separately or collectively dischargeable from the side supports 104 (e.g., where each side support has at least one release valve). Such a design is desirable in some scenarios because fluid can quickly be discharged from the side supports 104, which allows the human body situated on the pressure-mitigation device 100 to be accessed (e.g., in the case of a medical emergency). In other embodiments, fluid is only collectively dischargeable from the side supports 104. This approach to “dually deflating” the side supports 104 may be taken if release valve(s) are connected to only one side support, though both side supports are fluidically coupled to one another. The release valve(s) may be manually or electrically actuated. For example, the release valve(s) may be manually actuated by pressing a mechanical button (also referred to as a “strike button”) that, when pressed, allows fluid to flow out of the corresponding chamber or side support. In embodiments where the fluid is air, the air may be permitted to flow into the ambient environment. In embodiments where the fluid is water or gel, the fluid may be directed into a container (e.g., from which the fluid can then be rerouted through the controller as further discussed below). As another example, the release valve(s) may be electronically actuated by interacting with a switch assembly (e.g., located along the exterior surface of the pressure-mitigation device 100), a controller, or another computing device (e.g., a mobile phone or wearable electronic device) that is communicatively connected to the pressure-mitigation device 100.

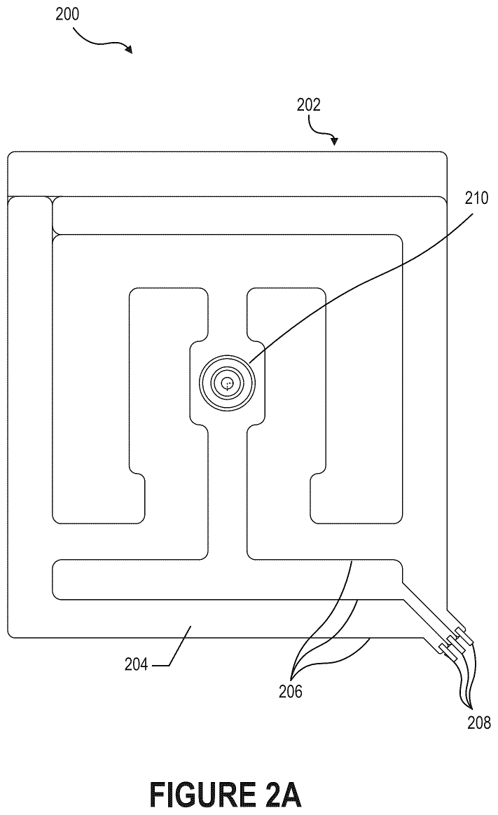

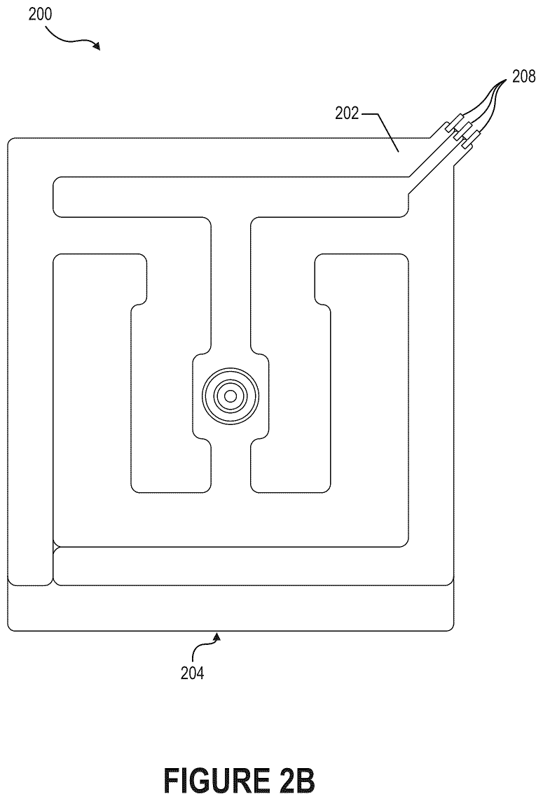

FIGS. 2A-B are top and bottom views, respectively, of a pressure-mitigation device 200 configured in accordance with embodiments of the present technology. The pressure-mitigation device 200 is generally used in conjunction with non-elongated objects that support individuals in a seated or partially erect position. Examples of non-elongated objects include chairs (e.g., office chairs, examination chairs, recliners, and wheelchairs) and the seats included in vehicles and airplanes. Accordingly, the pressure-mitigation device 200 may be positioned atop surfaces that have side supports integrated into the object itself (e.g., the side arms of a recliner or wheelchair). Note, however, that the pressure-mitigation device 200 could likewise be used in conjunction with elongated objects in a manner generally similar to the pressure-mitigation device 100 of FIGS. 1A-B.

In some embodiments, the pressure-mitigation device 200 is secured to a surface using an attachment apparatus. In other embodiments, the attachment apparatus is omitted such that the pressure-mitigation device 200 directly contacts the underlying surface. In such embodiments, the pressure-mitigation device 200 may have a tacky substance deposited along at least a portion of its outer surface that allows it to temporarily adhere to the surface.

The pressure-mitigation device 200 can include various features similar to the features of the pressure-mitigation device 100 described above with respect to FIGS. 1A-B. For example, the pressure-mitigation device 200 may include a first portion 202 (also referred to as a “first layer” or “bottom layer”) designed to face the surface, a second portion 204 (also referred to as a “second layer” or “top layer”) designed to face the human body supported by the surface, and a plurality of chambers 206 formed via interconnections between the first and second portions 202, 204. In this embodiment, the pressure-mitigation device 200 includes an “M-shaped” chamber intertwined with a backward “J-shaped” chamber and a backward “C-shaped” chamber. Varying the pressure in such an arrangement of chambers 206 has been shown to effectively mitigate the pressure applied by a surface to the gluteal and sacral regions of a human body in a seated position. These chambers may be intertwined to collectively form a square-shaped pattern. Pressure-mitigation devices designed for deployment on the surfaces of non-elongated objects may have substantially quadrilateral-shaped patterns of chambers, while pressure-mitigation devices designed for deployment on the surfaces of elongated objects may have substantially square-shaped patterns of chambers.

As further discussed below, the chambers 206 can be inflated and/or deflated in a predetermined pattern and to predetermined pressure levels. The individual chambers 206 may be inflated to higher pressure levels than the chambers 106 of the pressure-mitigation device 100 described with respect to FIGS. 1A-B because the human body being supported by the pressure-mitigation device 200 is in a seated position, thereby causing more pressure to be applied by the underlying surface than if the human body were in a supine or prone position. Further, unlike the pressure-mitigation device 100 of FIGS. 1A-1B, the pressure-mitigation device 200 of FIGS. 2A-2B does not include side supports. As noted above, side supports may be omitted when the object on which the individual is situated (e.g., seated or reclined) already provides components that will laterally center the human body, as is often the case with non-elongated support surfaces. One example of such a component is the armrests along the side of a chair.

As further described below (e.g., with respect to FIGS. 9A-9C), a controller can control the pressure in each chamber 206 by providing a discrete airflow via one or more corresponding valves 208. Here, the pressure-mitigation device 200 includes three valves 208, and each of the three valves 208 corresponds to a single chamber 206. Other embodiments of the pressure-mitigation device 200 may include fewer than three valves or more than three valves, and each valve can be associated with one or more chambers to control inflation/deflation of those chamber(s). A single valve could be in fluid communication with two or more chambers. Further, a single chamber could be in fluid communication with two or more valves (e.g., one valve for inflation and another valve for deflation).

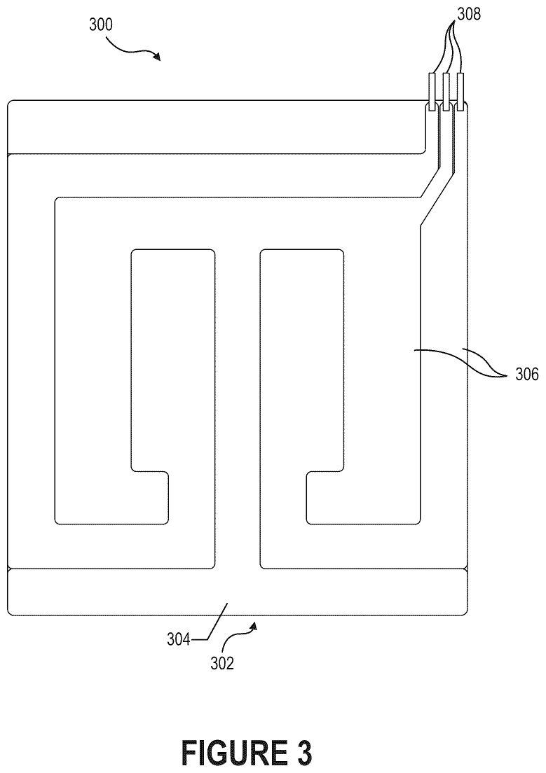

FIG. 3 is a top view of a pressure-mitigation device 300 for relieving pressure on an anatomical region applied by a wheelchair in accordance with embodiments of the present technology. The pressure-mitigation device 300 can include features similar to the features of the pressure-mitigation device 200 of FIGS. 2A-B and the pressure-mitigation device 100 of FIGS. 1A-1B described above. For example, the pressure-mitigation device 300 can include a first portion 302 (also referred to as a “first layer” or “bottom layer”) designed to face the seat of the wheelchair, a second portion 304 (also referred to as a “second layer” or “top layer”) designed to face the human body supported by the seat of the wheelchair, a series of chambers 306 formed by interconnections between the first and second portions 302, 304, and multiple valves 308 that control the flow of fluid into and/or out of the chambers 306. As can be seen in FIG. 3, the chambers 306 may be arranged similar to those shown in FIGS. 2A-2B. Here, however, the pressure-mitigation device 300 is designed such that the valves 308 will be located near the backrest of the wheelchair. Such a design may allow the tubing connected to the valves 308 to be routed through a gap near, beneath, or in the backrest.

In some embodiments the first portion 302 is directly adjacent to the seat of the wheelchair, while in other embodiments the first portion 302 is directly adjacent to an attachment apparatus. As shown in FIG. 3, the pressure-mitigation device 300 may include an “M-shaped” chamber intertwined with a “U-shaped” chamber and a “C-shaped” chamber, which are inflated and deflated in accordance with a predetermined pattern to mitigate the pressure applied to the sacral region of a human body in a sitting position on the seat of a wheelchair. These chambers may be intertwined to collectively form a square-shaped pattern.

According to the above descriptions, FIGS. 1A-3 describe pressure-mitigation devices that can be place atop substrates to mitigate pressure applied to human bodies or portions thereof. The term “substrate,” as used herein, may be used to refer to any underlying surface upon which a human body or a part thereof may be supported. Examples of substrates include beds (and more specifically, mattresses), chairs, wheelchairs, pillows, armrests, couches (and more specifically, cushions), backrests, neck-rests, headrests, and the like. Embodiments disclosed below integrate the pressure-mitigation mechanism of these devices into apparatuses that directly fit and/or secure to the substrates, or are integrated into the substrates themselves. As a result, embodiments disclosed below improve the pressure-mitigation treatment provided to patients, as slippage and displacement of the inflatable chambers providing the treatment is reduced.

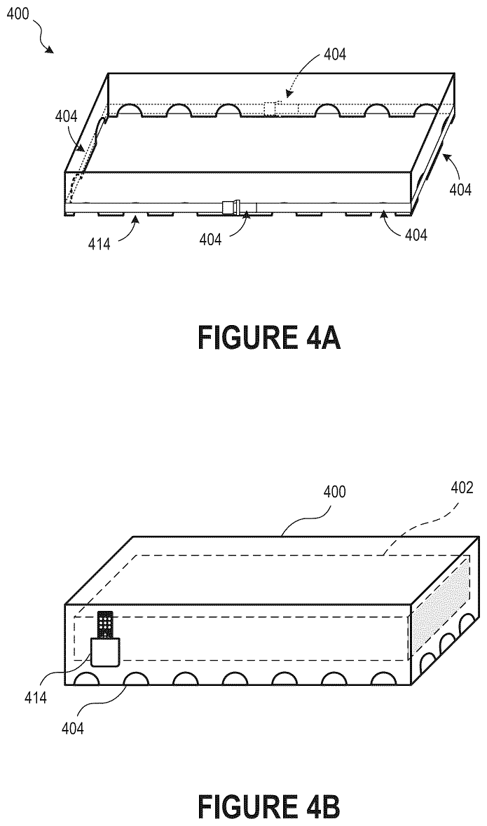

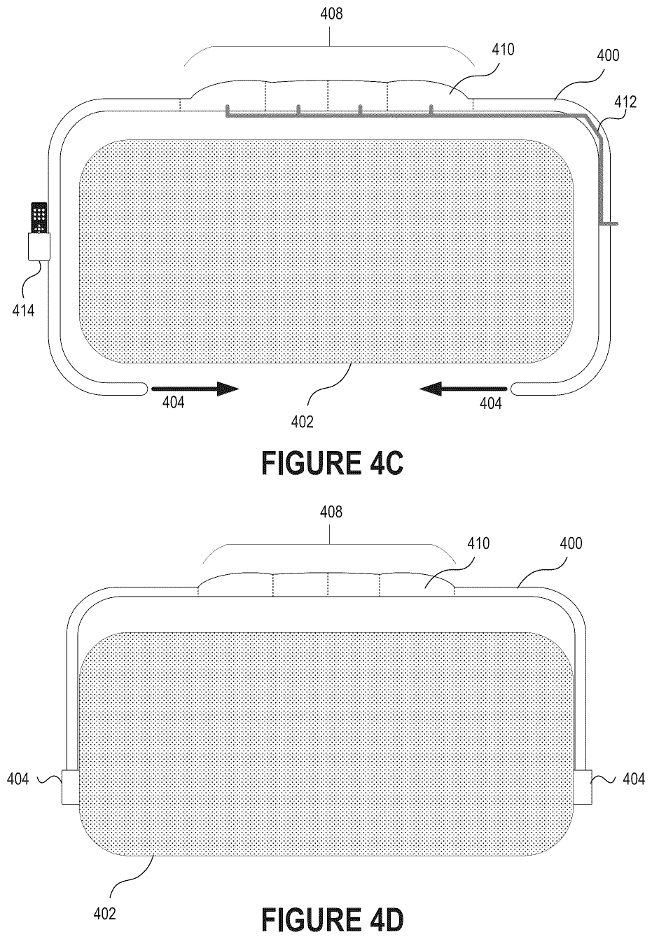

FIGS. 4A-4D illustrate various views of substrate-fitted pressure-mitigation devices, or apparatuses that incorporate pressure-mitigation mechanisms (e.g., including those disclosed above) while directly fitting and/or securing to substrates. These example embodiments may not rely upon material friction or separate equipment to maintain inflatable chambers in position to mitigate pressure exerted on a human body.

FIGS. 4A and 4B illustrate an example pressure-mitigation system that includes a pressure-mitigation cover 400 that is configured to fit and/or fasten to a substrate 402, such as a mattress, cushion, pad, pillow, and/or the like. In some embodiments, the pressure-mitigation cover 400 is constructed from a flexible fabric material that is overlapped and folded to form a three-dimensional construction. For example, based on folding at corners of the pressure-mitigation cover 400, the pressure-mitigation cover 400 substantially includes a planar portion or region and side portions or regions that contiguously extend out of a plane of the planar portion/region. As such, the pressure-mitigation cover 400 can define a cavity in which the substrate 402 fits, as illustrated in FIG. 4B. More particularly, the cavity can include a maximum volume that is greater than a volume of an intended substrate. In the illustrated examples of FIGS. 4A and 4B, the cavity is specifically defined by the planar portion/region and four side portions.

While FIGS. 4A and 4B illustrate the pressure-mitigation cover 400 having a substantially a rectangular prism shape, with four side portions or regions that each extend down one of four sidewalls of the substrate 402, it will be understood that different embodiments of the pressure-mitigation cover 400 can be configured with different shapes. For example, the pressure-mitigation cover 400 includes less than four side portions. In some examples, the pressure-mitigation cover 400 resembles a sleeve or sock, having an upper cover portion, a bottom cover portion, and a number of side portions that is less than a number of sidewalls of the substrate (e.g., one, two, three when the substrate is substantially rectangular). In some examples, a pressure-mitigation cover 400 configured as a sleeve includes an upper cover portion that does not span the entire area of the upper surface of the substrate and resembles a band when fitted on the substrate. In some examples, the side portions of the pressure-mitigation cover 400 extend longitudinally along a length of the pressure-mitigation cover 400 to drape down the sides of the substrate. In some examples, the side portions of the pressure-mitigation cover 400 extend latitudinally along a width of the pressure-mitigation cover 400 to drape down front and back sidewalls of the substrate. In some examples in which the substrate is rectangular, it will be appreciated that a pressure-mitigation cover 400 having latitudinally extending side portions (in lieu of longitudinally extending side portions) conserves an amount of construction material. In some examples, the pressure-mitigation cover 400 includes one side portion that extends latitudinally and drapes down a front or a back sidewall of the substrate. In such examples, the pressure-mitigation cover 400 having one side portion accounts for a frame or structure used with the substrate, such as a headboard (for a mattress substrate), a seatback (for a seat cushion substrate), and/or the like.

In other embodiments, each portion or region of the pressure-mitigation cover 400 is a separate panel, and the portions or regions of the pressure-mitigation cover 400 are attached (e.g., sewed, molded, zippered) to one another at the edges illustrated in FIGS. 4A and 4B. Generally then, the pressure-mitigation cover can include an upper cover portion, and side cover portions that extend from the upper cover portion and down sidewalls of the substrate. In some embodiments, the pressure-mitigation cover 400 is a single-panel construction and is a continuous panel having upper and side portions or regions.

FIGS. 4A and 4B further illustrate fastening mechanisms 404 included in the pressure-mitigation cover 400 that facilitate the fit and/or fastening of the pressure-mitigation cover 400 to and/or within the substrate 402. In some embodiments, the fastening mechanisms include an elastic band that spans through the bottom edge or portion of the side portions/regions of the pressure-mitigation cover 400. The elastic band can then constrict the side cover portions to and/or below the sides of the substrate 402. In some embodiments, the fastening mechanisms include a band (e.g., the elastic band), strap, string, rope, and/or the like that can be tightened via a tensioner (e.g., a ratch tensioner). Similarly, with the tensioner, the side cover portions can be constricted to and/or below the sides of the substrate 402. Accordingly, the improved fit of the pressure-mitigation cover 400 to the substrate 402 can comprehensively span multiple sides of the substrate 402. In some embodiments, the band, strap, string, rope, and/or the like extends within a channel sewed or constructed within the side portions/regions of the pressure-mitigation cover 400.

The pressure-mitigation cover 400 can include other various fastening mechanisms. In particular, in some embodiments, the pressure-mitigation cover 400 includes a fastening mechanism that engages, interfaces, attaches, and/or the like with a feature on the substrate 402. For example, the pressure-mitigation cover 400 can include hook-and-loop fasteners that secure to corresponding hook-and-loop fasteners located on the substrate 402 (e.g., on the sides and/or bottom of the substrate 402). As another example, a bottom edge of a side cover portion of the pressure-mitigation cover 400 can include a zipper that can be secured with a zipper located on the substrate 402 (e.g., on the sides and/or bottom of the substrate 402). As yet another example, the pressure-mitigation cover 400 can include straps that can tie and/or loop through corresponding straps, holes, or features on the substrate. As yet another example, the pressure-mitigation cover 400 can include buttons that interface with and secure to features on the substrate. Generally, the fastening mechanisms of the pressure-mitigation cover 400 can include hooks, tabs, perforations, straps, and/or the like. As yet another example, the pressure-mitigation cover 400 can include magnets that are magnetically attracted to corresponding magnets incorporated into the substrate. As yet another example, the pressure-mitigation cover 400 includes weights or weighted sections (e.g., a pocket of weighted beads, a pocket including a metal weight) that sit within a depression in the substrate. Specifically, the pressure-mitigation cover 400 does not include side portions and resembles a blanket having weights at the edges that sit in specific depressions, holes, cavities, rings, and/or the like in an upper surface of the substrate.

In some embodiments, the fastening mechanisms are configured to secure the pressure-mitigation cover 400 to the substrate via associated structures of the substrate. For example, the fastening mechanism is a flexible strap that wraps around a bar of a substrate frame (e.g., a bed frame, a bed headboard, a chair frame). As another example, the fastening mechanism includes a magnet that is magnetically attracted to at least a portion of the substrate frame.

FIGS. 4C and 4D provide partially sectional views of the pressure-mitigation system that includes a pressure-mitigation cover 400 and a substrate 402. For example, the pressure-mitigation cover 400 illustrated in FIGS. 4C and 4D envelops and covers the substrate 402, and specifically, the upper surface of the substrate 402 on which a human body can be disposed. As illustrated, the pressure-mitigation cover 400 includes or is configured with fastening mechanisms 404. In FIG. 4C, the fastening mechanism 404 causes portions of the pressure-mitigation cover 400 to constrict to/below the substrate 402, such that the substrate 402 is securely enveloped and disposed within the pressure-mitigation cover 400. In FIG. 4D, the fastening mechanism 404 attaches to a feature of the substrate 402. While FIG. 4D demonstrates the pressure-mitigation cover 400 being fastened to the sides of the substrate 402, it will be understood that, in various embodiments, the fastening mechanism can attach to features located on the bottom, sides, or top surface of the substrate 402.

In particular, FIGS. 4C and 4D demonstrate the pressure-mitigation mechanism that is incorporated and integrated within the pressure-mitigation cover 400 (not explicitly shown in FIGS. 4A and 4B). As shown, the pressure-mitigation cover 400 can include a pressure-mitigation region/portion 408 that is disposed atop the upper surface of the substrate 402 when the substrate 402 is enveloped by or disposed within a cavity of the pressure-mitigation cover 400. For example, the pressure-mitigation region/portion 408 is included in the upper or planar portion/region of the pressure-mitigation cover 400. In some embodiments, the pressure-mitigation region/portion 408 spans at most the entire surface area of the upper surface of the substrate 402. In the illustrated example, the pressure-mitigation region/portion 408 spans less than the entire surface area of the upper surface of the substrate 402.

The pressure-mitigation region/portion 408 can include one or more inflatable chambers 410. In some embodiments, the inflatable chambers 410 are configured according to embodiments disclosed with respect to FIGS. 1A-3. That is, the pressure-mitigation region/portion 408 can resemble the pressure-mitigation devices of FIGS. 1A-3, or the pressure-mitigation devices of FIGS. 1A-3 can be incorporated or integrated as the pressure-mitigation region/portion 408 of the pressure-mitigation cover 400. As illustrated in FIGS. 4C and 4D, the inflatable chambers 410 are defined by interconnections, bulkheads, walls, and/or the like between a top surface and a bottom surface of the pressure-mitigation cover 400. In some embodiments, the fastening mechanism 404 further includes tacky or non-slip material being included on the bottom surface of the pressure-mitigation cover 400 (e.g., at least within the pressure-mitigation region/portion 408), such that the tacky or non-slip material faces the upper surface of the substrate 402.

In including the pressure-mitigation region/portion 408, the pressure-mitigation cover 400 can include tubing 412 that transports fluid to and from the inflatable chambers 410. In some embodiments, similar to a fastening strap or elastic band spanning through a channel defined in the pressure-mitigation cover 400, the tubing 412 for the inflatable chambers 410 extend through channels defined in the pressure-mitigation cover 400. For example, tubing channels are constructed within the side portions of the pressure-mitigation cover 400, and the pressure-mitigation cover 400 includes tubing attachment ports towards the bottom of the side portions (e.g., ends of the side portions opposite of the upper or planar portion). As such, critical attachment and joint points are distanced away from the upper surface of the substrate 402 and any human bodies disposed thereupon. The illustrated example demonstrates the tubing 412 extending through a bottom layer of the inflatable chambers 410. In other examples, the tubing 412 extends laterally from the inflatable chambers 410 (e.g., in and out of a depth of the page of FIG. 4C).

In some embodiments, the pressure-mitigation cover 400 includes a pocket 414 that is configured to hold a controller that operates the inflatable chambers 410, such as the controllers disclosed herein. Specifically, in some embodiments, the pocket 414 is located on a side cover portion and is configured to face “upward” or towards the upper or planar cover portion when the pressure-mitigation cover 400 is in position atop and enveloping the substrate 402. In some embodiments, the controller is integrated into the pressure-mitigation cover 400. For example, the pressure-mitigation cover 400 includes a closed pocket in which the controller is disposed, and the closed pocket includes a transparent window (e.g., a flexible plastic or vinyl window) via which an individual can view and interact with a user interface of the controller. In such an example, the closed pocket may be sewn shut, or the closed pocket includes a closure mechanism (e.g., hook-and-loop fasteners, zippers, buttons) to enclose the controller within the pressure-mitigation cover 400.

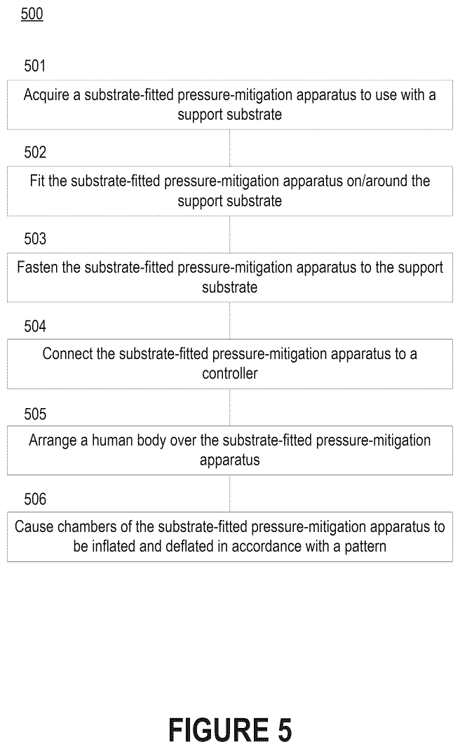

FIG. 5 is a flow diagram of a process 500 for deploying a pressure-mitigation system designed to prevent and/or address ischemia-reperfusion injuries in accordance with embodiments of the present technology. The pressure-mitigation system can be configured to maintain a pressure-mitigation mechanism (e.g., inflatable chambers) underneath a target region of a human body with reduced slippage and displacement. Initially, an individual can acquire a substrate-fitted pressure-mitigation apparatus to use with a support substrate (step 501). The support substrate can have a support surface, and the substrate-fitted pressure-mitigation apparatus can include inflatable chambers that, when in use, are disposed between a human body and the support surface. The individual may be the person who will be treated by the pressure-mitigation system or some other person (e.g., a physician, nurse, or caregiver). In some embodiments, the individual selects the pressure-mitigation apparatus from amongst multiple pressure-mitigation apparatuses designed for different body types, anatomical regions, or support surfaces.

The individual can then fit the substrate-fitted pressure-mitigation apparatus on and/or around the support substrate (step 502). In some examples, this may include laying the substrate-fitted pressure-mitigation apparatus atop the support substrate, manipulating the substrate-fitted pressure-mitigation apparatus such that the support substrate is disposed within a cavity of the substrate-fitted pressure-mitigation apparatus, sliding the substrate into the substrate-fitted pressure-mitigation apparatus, and/or the like.

The individual can further fasten the substrate-fitted pressure-mitigation apparatus to the support substrate (step 503). In particular, the substrate-fitted pressure-mitigation apparatus can include fastening mechanisms to augment the fit of the substrate-fitted pressure-mitigation apparatus to the support substrate. As an example, while a pressure-mitigation mattress cover is dimensioned to fit a mattress, the pressure-mitigation mattress cover further include an elastic band, straps, buttons, fasteners, and/or the like to further attach to the mattress. Here, in particular, the individual uses fastening mechanisms that require active manipulation and use, such as manipulating a button, engaging a zipper, and/or the like. In some embodiments, the substrate-fitted pressure-mitigation apparatus includes passive fastening mechanisms that fulfill the fastening (of step 503) as a result of the substrate-fitted pressure-mitigation apparatus being fitted on/around the substrate. For example, an interior surface (e.g., facing the substrate) of the substrate-fitted pressure-mitigation apparatus can be comprised of at least one material that provides some tackiness, such as silicone rubber, to naturally limit movement. In some embodiments, the interior surface of the substrate-fitted pressure-mitigation apparatus includes an adhesive film with sufficient tackiness to limit movement through more permanent adhesion. In such embodiments, the individual may need to remove a cover or a film from the interior surface of the attachment apparatus before securing the substrate-fitted pressure-mitigation apparatus to the support substrate.

The individual can then connect the pressure-mitigation apparatus to a controller (step 504). For example, the individual may fluidly couple the controller to the pressure-mitigation apparatus using multi-channel tubing. In some embodiments, the individual connects the controller with tubing that extends through at least a portion of the substrate-fitted pressure-mitigation apparatus. In some embodiments, the controller may be configured to automatically determine whether a pressure-mitigation apparatus has been connected.

Thereafter, the individual can arrange a human body over the substrate-fitted pressure-mitigation apparatus for the human body to be treated (step 505). The substrate-fitted pressure-mitigation apparatus may include a geometric pattern of chambers designed to mitigate the pressure on a specific anatomical region of the human body. Accordingly, the human body may need to be oriented over a particular region (also referred to as a “target region”) of the substrate-fitted pressure-mitigation apparatus. In some embodiments, the target region may be visually distinguishable along the upper surface of the pressure-mitigation apparatus. Due to the fitting and/or fastening of the substrate-fitted pressure-mitigation apparatus (e.g., at steps 502 and 503), movement of the substrate-fitted pressure-mitigation apparatus is reduced while the individual arranges the human body over the substrate-fitted pressure-mitigation apparatus.

The individual can operate the controller to cause the chambers of the substrate-fitted pressure-mitigation apparatus to be inflated in accordance with a pattern (step 506). More specifically, the controller can cause the pressure on anatomical region(s) of the human body to be varied by controllably inflating chamber(s), deflating chamber(s), or any combination thereof. The pattern may correspond to the pressure-mitigation apparatus. For example, upon detecting that a given pressure-mitigation apparatus has been connected to the controller, the controller may examine a library of patterns corresponding to different pressure-mitigation apparatuses having different counts/arrangements of chambers to identify the appropriate pattern. Inflation (and deflation) of the chambers result in the respective heights of the chambers to dynamically change relative to other chambers and relative to the substrate-fitted pressure-mitigation apparatus.

Unless contrary to physical possibility, it is envisioned that the steps described above may be performed in various sequences and combinations. For example, the individual may fasten the substrate-fitted pressure-mitigation apparatus to the support substrate before fitting the substrate-fitted attachment apparatus to the support substrate. As another example, the individual may connect the substrate-fitted pressure-mitigation apparatus to the controller before fastening the substrate-fitted pressure-mitigation apparatus to the support substrate. In some embodiments, a controller is integrated into the substrate-fitted pressure-mitigation apparatus, and at step 504, the individual instead connects the substrate-fitted pressure-mitigation apparatus to a fluid supply device. Other steps may also be included in some embodiments. For example, before causing the chambers of the pressure-mitigation apparatus to be inflated in accordance with the pattern, the controller may prompt an operator to specify a characteristic of the human body to be treated by the pressure-mitigation system, such as the size, weight, degree of immobility, or position.

### Overview of Approaches to Mitigating Pressure

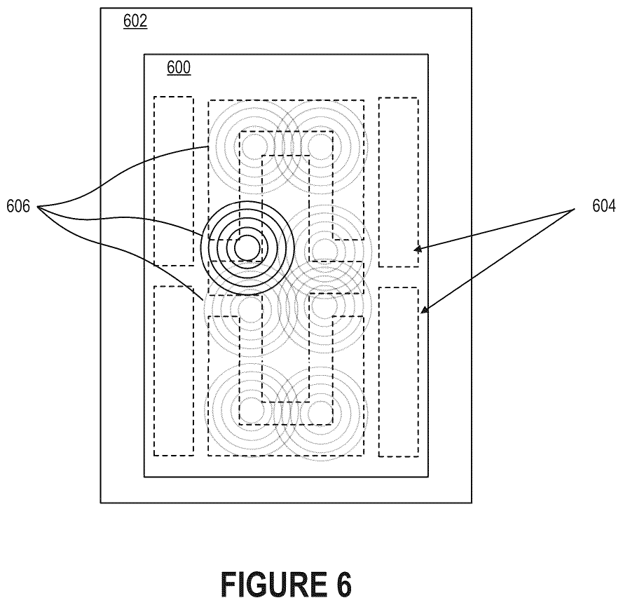

FIG. 6 is a partially schematic top view of a pressure-mitigation device, for example integrated into a substrate or a substrate cover, illustrating how a pressure gradient can be created by varying pressure distributions to avoid ischemia in a mobility-impaired patient in accordance with embodiments of the present technology. When a human body is supported by a surface 602 of a substrate for an extended duration, pressure injuries may form in the tissue overlaying bony prominences, such as the skin overlying the sacrum, coccyx, heels, or hips. Generally, these bony prominences represent the locations at which the most pressure is applied by the surface 602 and, therefore, may be referred to as the “main pressure points” along the surface of the human body.

To prevent the formation of pressure injuries, healthy individuals periodically make minor positional adjustments (also known as “micro-adjustments”) to shift the location of the main pressure point. However, individuals having impaired mobility often cannot make these micro-adjustments by themselves. Mobility impairment may be due to physical injury (e.g., a traumatic injury or a progressive injury), movement limitations (e.g., within a vehicle, on an aircraft, or in restraints), medical procedures (e.g., those requiring anesthesia), and/or other conditions that limit natural movement. For these mobility-impaired individuals, the pressure-mitigation device 600 can be used to shift the location of the main pressure point(s) on their behalf. That is, the pressure-mitigation device 600 can create moving pressure gradients to avoid sustained, localized vascular compression and enhance tissue perfusion.

The pressure-mitigation device 600 can include a series of chambers 604 whose pressure can be individually varied. The chambers 604 may be formed by interconnections between the top and bottom layers of the pressure-mitigation device 600. The top layer may be comprised of a first material (e.g., a permeable, non-irritating material) configured for direct contact with a human body, while the bottom layer may be comprised of a second material (e.g., a non-permeable, gripping material) configured for direct contact with the surface 602. Generally, the first material is permeable to gasses (e.g., air) and/or liquids (e.g., water and sweat) to prevent buildup of fluids that may irritate the skin. Meanwhile, the second material may not be permeable to gasses or liquids to prevent soilage of the underlying object. Accordingly, air discharged into the chambers 604 may be able to slowly escape through the first material (e.g., naturally or via perforations) but not the second material, while liquids may be able to penetrate the first material (e.g., naturally or via perforations) but not the second material. Note, however, that the first material is generally be selected such that the top layer does not actually become saturated with liquid to reduce the likelihood of irritation. Instead, the top layer may allow liquid to pass therethrough into the cavities, from which the liquid can be subsequently discharged (e.g., as part of a cleaning process). The top layer and/or the bottom layer can be comprised of more than one material, such as a coated fabric or a stack of interconnected materials.

The pressure-mitigation device 600 may be designed such that inflation of at least some of the chambers 604 causes air to be continuously exchanged across the surface of the human body. Said another way, simultaneous inflation of at least some of the chambers 604 may provide a desiccating effect to inhibit generation and/or collection of moisture along the skin in a given anatomical region. In some embodiments, the pressure-mitigation device 600 is able to maintain airflow through the use of a porous material. For example, the top layer may be comprised of a biocompatible material through which air can flow (e.g., naturally or via perforations). In other embodiments, the pressure-mitigation device 600 is able to maintain airflow without the use of a porous material. For example, airflows can be created and/or permitted simply through varied pressurization of the chambers 604. This represents a new approach to microclimate management that is enabled by simultaneous inflation and deflation of the chambers 604. At a high level, each void formed beneath a human body due to deflation of at least one chamber can be thought of as a microclimate that cools and desiccates the corresponding portion of the anatomical region. Heat and humidity can lead to injury (e.g., further development of ulcers), so the cooling and desiccating effects may present some injuries due to inhibition of moisture generation/collection along the skin in the anatomical region.

In some embodiments, a pump (also referred to as a “pressure device”) can be fluidically coupled to each chamber 604 (e.g., via a corresponding valve) of each pressure-mitigation device, while a controller can control the flow of fluid generated by the pump into each chamber 604 on an individual basis in accordance with a predetermined pattern. The controller can operate the series of chambers 604 in several different ways.

In some embodiments, the chambers 604 have a naturally deflated state, and the controller causes the pump to inflate at least one of the chambers 604 to shift the main pressure point along the anatomy of the human body. For example, the pump may inflate at least one chamber located directly beneath an anatomical region to momentarily apply contact pressure to that anatomical region and relieve contact pressure on the surrounding anatomical regions adjacent to the deflated chamber(s). Alternatively, the controller may cause the pump to inflate two or more chambers adjacent to an anatomical region to create a void beneath the anatomical region to shift the main pressure point at least momentarily away from the anatomical region.

In other embodiments, the chambers 604 have a naturally inflated state, and the controller may cause deflation of at least one of the chambers 604 to shift the main pressure point along the anatomy of the human body. For example, the pump may cause deflation of at least one chamber located directly beneath an anatomical region, thereby forming a void beneath the anatomical region to momentarily relieve the contact pressure on the anatomical region. To deflate a chamber, the controller may simply prevent an airflow generated by the pump from entering the chamber as further discussed below. Additionally or alternatively, the controller may cause air contained in the chamber to be released (e.g., via a valve). At least partial deflation may naturally occur in this scenario if air escapes through the valve quicker than air enters the chamber.

Whether configured in a naturally deflated state or a naturally inflated state, the continuous or intermittent alteration of the inflation levels of the individual chambers 604 moves the location of the main pressure point across different portions of the human body. As shown in FIG. 6, for example, inflating and/or deflating the chambers 604 creates temporary contact regions 606 that move across the pressure-mitigation device 600 in a predetermined pattern, and thereby changing the location of the main pressure point(s) on the human body for finite intervals of time. Thus, the pressure-mitigation device 600 can simulate the micro-adjustments made by healthy individuals to relieve stagnant pressure applied by the surface 602.

The series of chambers 604 may be arranged in an anatomy-specific pattern so that when the pressure of one or more chambers is altered, the contact pressure on a specific anatomical region of the human body is relieved (e.g., by shifting the main pressure point elsewhere). As an example, the main pressure point may be moved between eight different locations corresponding to the eight temporary contact regions 606 as shown in FIG. 6. In some embodiments the main pressure point shifts between these locations in a predictable manner (e.g., in a clockwise or counter-clockwise pattern), while in other embodiments the main pressure point shifts between these locations in an unpredictable manner (e.g., in accordance with a random pattern or a semi-random pattern, based on the amount of force applied by the human body to the chambers, or based on the pressure of the chambers). Those skilled in the art will recognize that the number and position of these temporary contact regions 606 may vary based on the size of the pressure-mitigation device 600, the arrangement of chambers 604, the number of chambers 604, the anatomical region supported by the pressure-mitigation device 600, the characteristics of the human body supported by the pressure-mitigation device 600, the condition of the human body (e.g., whether the person is completely immobilized, partially immobilized, etc.), or any combination thereof.

As discussed above, the pressure-mitigation device 600 may not include side supports if the condition of a user (also referred to as the “patient” or “subject”) would not benefit from the positioning assistance provided by the side supports. For example, side supports can be omitted when the user is medically immobilized (e.g., under anesthesia, in a medically induced coma, etc.) and/or physically restrained on the underlying surface 602 (e.g., by rails on the side of a bed, arm rests on the side of a chair, restraints that limit movement, etc.).

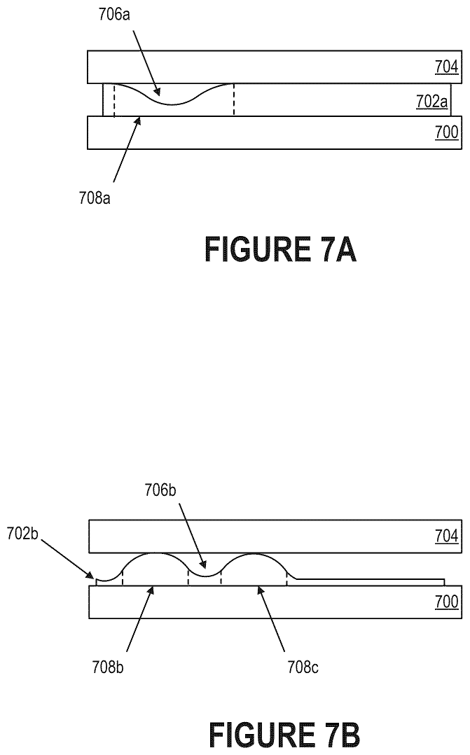

FIG. 7A is a partially schematic side view of a pressure-mitigation device, such as an alignment-facilitating device 702a, for relieving pressure on a specific anatomical region by deflating one or more chambers in accordance with embodiments of the present technology. The pressure-mitigation device 702a can be positioned between the surface of an object 700 and a human body 704. Examples of objects 700 include elongated objects, such as mattresses, stretchers, operating tables, and procedure tables, and non-elongated objects, such as chairs (e.g., office chairs, examination chairs, recliners, and wheelchairs) and the seats included in vehicles and airplanes. To relieve the pressure on a specific anatomical region of the human body 704, at least one chamber 708a of multiple chambers (collectively referred to as “chambers 708”) proximate to the specific anatomical region is at least partially deflated to create a void 706a beneath the specific anatomical region. In such embodiments, the remaining chambers 708 may remain inflated. Thus, the pressure-mitigation device 702a may sequentially deflate chambers (or arrangements of multiple chambers) to relieve the pressure applied to the human body 704 by the surface of the object 700.

FIG. 7B is a partially schematic side view of a pressure-mitigation device 702b for relieving pressure on a specific anatomical region by inflating one or more chambers in accordance with embodiments of the present technology. For example, to relieve the pressure on a specific anatomical region of the human body 704, the pressure-mitigation device 702b can inflate two chambers 708b and 708c disposed directly adjacent to the specific anatomical region to create a void 706b beneath the specific anatomical region. In such embodiments, the remaining chambers may remain partially or entirely deflated. Thus, the pressure-mitigation device 702b may sequentially inflate a chamber (or arrangements of multiple chambers) to relieve the pressure applied to the human body 704 by the surface of the object 700.

The pressure-mitigation devices 702a, 702b of FIGS. 7A-B are shown to be in direct contact with the contact surface. However, in some embodiments, an attachment apparatus is positioned between the pressure-mitigation devices 702a, 702b and the object 700. The attachment apparatus may be designed to help secure the pressure-mitigation devices 702a, 702b and the object 700. For example, the attachment apparatus may be made of a material that is naturally tacky or sticky so as to inhibit movement of the pressure-mitigation devices 702a, 702b with respect to the object 700. Alternatively, the bottom side of the pressure-mitigation devices 702a, 702b could be coated with a material, such as a removable adhesive (e.g., an elastomer- or silicone-based sealant or a pressure-sensitive film) or tacky substance (e.g., silicone rubber).

In some embodiments, the pressure-mitigation devices 702a, 702b of FIGS. 7A-B have the same configuration of chambers 708 and can operate in both a normally inflated state (described with respect to FIG. 7A) and a normally deflated state (described with respect to FIG. 7B) based on the selection of an operator (e.g., the user or some other person, such as a medical professional or family member). For example, the operator can use a controller to select a normally deflated mode such that the pressure-mitigation device operates as described with respect to FIG. 7B, and then change the mode of operation to a normally inflated mode such that the pressure-mitigation device operates as described with respect to FIG. 7A. Thus, the pressure-mitigation devices described herein can shift the location of the main pressure point by controllably inflating chambers, controllably deflating chambers, or a combination thereof.

While the embodiments disclosed above involve a separate pressure-mitigation device (e.g., pressure-mitigation devices 702a, 702b) being placed atop a contact surface of a substrate (e.g., object 700), embodiments disclosed below directly integrate the pressure-mitigation device with the substrate. By way of the following embodiments, an amount of equipment needed to perform the pressure-mitigation treatment can be reduced. The unitary pressure-mitigation substrate can then be a portable unit and can be transported to different locations. For example, a unitary pressure-mitigation pad can be used by a patient in many different environments (e.g., on bleachers at a sporting event and at home on a chair).

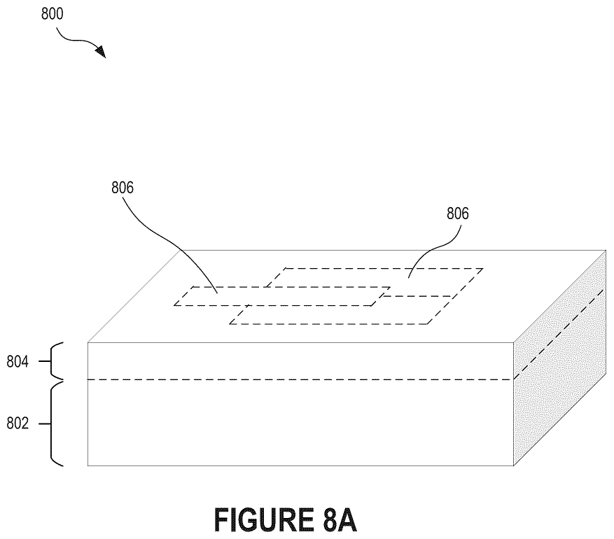

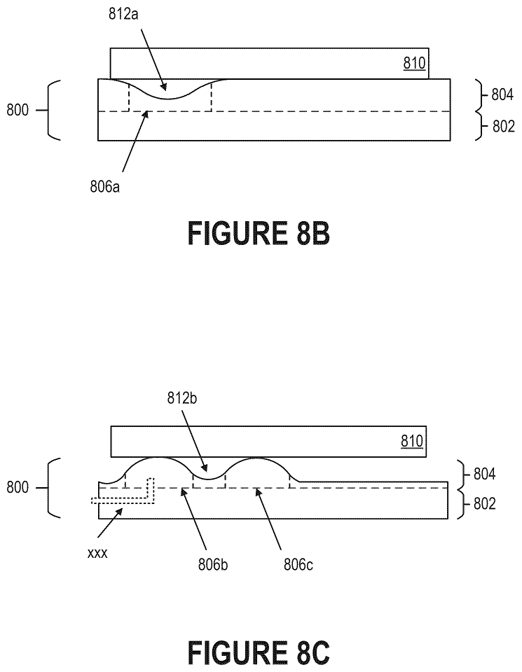

FIGS. 8A-8C illustrate various views of unitary or substrate-integrated pressure-mitigation apparatuses 800. These example pressure-mitigation apparatuses are mattresses, cushions, pads, pillows, seats, rests and/or the like that directly incorporate pressure-mitigation mechanisms as disclosed herein. FIG. 8A provides a perspective view that shows a pressure-mitigation apparatus 800 (e.g., a mattress) having a foundation layer 802 and a pressure-mitigation layer 804. The foundation layer and the pressure-mitigation layer can be stacked and together form the pressure-mitigation apparatus. The foundation layer 802 can provide a base structure for the pressure-mitigation layer 804, and the pressure-mitigation layer 804 can be stacked atop the foundation layer 802. In particular, the foundation layer 802 can be capable of supporting or sustaining a weight of at least a portion of a human body atop the pressure-mitigation apparatus 800. For example, the foundation layer 802 is a pad foundation. Example pad foundations include mattress box springs, mattress bases, cushion base layers, mattress base layers (support foam layer, a spring layer), and/or the like. Generally, layers or vertically-arranged portions of the pressure-mitigation apparatuses 800 that support the pressure-mitigation layer 804 may be considered as a foundation layer 802. As illustrated, the pressure-mitigation layer 804 can include one or more inflatable chambers 806 configured according to pressure-mitigation devices disclosed herein to spread the pressure exerted onto a human body disposed atop the pressure-mitigation apparatus 800.

While FIG. 8A illustrates the foundation layer and the pressure-mitigation layer, it will be understood that other example pressure-mitigation apparatuses can include one or more other layers, such as spring or coil layers, foam layers, cushioning layers, transition layers, comfort layers, and/or the like. In such examples, the pressure-mitigation layer 804 can remain the top layer of the stack, or closer to the top of the stack.

In some embodiments, the inflatable chambers 806 of the pressure-mitigation layer 804 are defined by bulkheads, walls, interconnections, and/or the like located within the pressure-mitigation layer 804. Such bulkheads, walls, interconnections, and/or the like can span between a top and a bottom of the pressure-mitigation layer 804. In some embodiments, as illustrated, the inflatable chambers 806 occupy less than a total area spanned by the pressure-mitigation layer 804.

As discussed above, incorporation of pressure-mitigation mechanisms into a substrate to result in a unitary pressure-mitigation substrate can facilitate portability of pressure-mitigation treatment and improve applicability and use in different environments. In some embodiments, the pressure-mitigation apparatus 800 includes handles, straps, and/or the like that facilitate transport of the pressure-mitigation apparatus 800. In particular, the handles, straps, and/or the like facilitate transport of the pressure-mitigation apparatus 800 without separation of the layers. For example, the ends of the handles are attached to different layers. As another example, the multiple layers are encased, and the handles are located on the casing or encasement.

Turning to FIGS. 8B and 8C, partial sectional views of the pressure-mitigation apparatus 800 are shown. For example, FIGS. 8B and 8C show a human body 810 being disposed atop the pressure-mitigation apparatus 800. The pressure-mitigation apparatus 800 includes a foundation layer 802 and a pressure-mitigation layer 804, and the pressure-mitigation layer 804 includes one or more chambers 806. In some embodiments, the chambers 806 can be deflated to create voids 812 between the pressure-mitigation apparatus 800 and the human body 810. Inversely, the chambers 806 can be inflated to also create voids 812 (e.g., void 812b) between the pressure-mitigation apparatus 800 and the human body 810. Precisely, deflation and inflation of the chambers 806 results in heights of the chambers 806 being dynamically varied relative to a top of the pressure-mitigation layer 804.

For the inflation and deflation of the chambers 806, the pressure-mitigation apparatus 800 may include tubing that transports fluid to and from the chambers 806. In some embodiments, the tubing meets the chambers 806 from the bottom of the chambers 806 (e.g., the bottom of the pressure-mitigation layer 804), and in some embodiments, the tubing extends through one or more lower layers of the pressure-mitigation apparatus 800. For example, a tubing layer that provides a channeled structure through which the tubing extends is located directly underneath the pressure-mitigation layer 804, and sides of the tubing layer include tubing attachment ports via which pressure devices (e.g., fluid supplies) can be connected with the tubing. In doing so, critical attachment points and joints and kept away from the upper surface where a human body is located.

### Overview of Controller Devices

FIGS. 9A-9C are isometric, front, and back views, respectively, of a controller 900 (also referred to as a “controller device”) that is responsible for controlling inflation and/or deflation of the chambers of pressure-mitigation devices in accordance with embodiments of the present technology. For example, the controller 900 can be coupled to pressure-mitigation devices to control the pressure within the chambers of the pressure-mitigation devices. The controller 900 can manage the pressure in each chamber of the pressure-mitigation devices by controllably driving one or more pumps. In some embodiments, a single pump is fluidically connected to all the chambers of the two or more pressure-mitigation devices, such that the pump is responsible for independently directing fluid flow to and/or from multiple chambers. In other embodiments, the controller 900 is coupled to two or more pumps, each of which can be fluidically coupled to a single chamber to drive inflation/deflation of that chamber. In other embodiments, the controller 900 is coupled to at least one pump that is fluidically coupled to two or more chambers and/or at least one pump that is fluidically coupled to a single chamber. The pump(s) may reside within the housing of the controller 900 such that the system is easily transportable. Alternatively, the pump(s) may reside in a housing separate from the controller 900.

As shown in FIGS. 9A-9C, the controller 900 can include a housing 902 in which internal components reside and a handle 904 that is connected to the housing 902. In some embodiments the handle 904 is fixedly secured to the housing 902 in a predetermined orientation, while in other embodiments the handle 904 is pivotably secured to the housing 902. For example, the handle 904 may be rotatable about a hinge connected to the housing 902 between multiple positions. The hinge may be one of a pair of hinges connected to the housing 902 along opposing lateral sides. The handle 904 enables the controller 900 to be readily transported, for example, from a storage location to a deployment location (e.g., proximate a human body that is positioned on a surface). Moreover, the handle 904 could be used to releasably attach the controller 900 to a structure. For example, the handle 904 could be hooked on an intravenous (IV) pole (also referred to as an “IV stand” or “infusion stand”).

In some embodiments, the controller 900 includes a retention mechanism 914 that is attached to, or integrated within, the housing 902. Cords (e.g., electrical cords), tubes, and/or other elongated structures associated with the system can be wrapped around or otherwise supported by the retention mechanism 914. Thus, the retention mechanism 914 may provide strain relief and retention of an electrical cord (also referred to as a “power cord”). In some embodiments, the retention mechanism 914 includes a flexible flange that can retain the plug of the electrical cord.

As further shown in FIGS. 9A-9C, the controller 900 may include a connection mechanism 912 that allows the housing 902 to be securely, yet releasably, attached to a structure. Examples of structures include IV poles, mobile workstations (also referred to as “mobile carts”), bedframes, rails, handles (e.g., of wheelchairs), and tables. The connection mechanism 912 may be used instead of, or in addition to, the handle 904 for mounting the controller 900 to the structure. In the illustrated embodiment, the connection mechanism 912 is a mounting hook that allows for single-hand operation and is adjustable to allow for attachment to mounting surfaces with various thicknesses. In some embodiments, the controller 900 includes an IV pole clamp 916 that eases attachment of the controller 900 to IV poles. The IV pole clamp 916 may be designed to enable quick securement, and the IV pole clamp 916 can be self-centering with the use of a single activation mechanism (e.g., knob or button).

In some embodiments, the housing 902 includes one or more input components 906 for providing instructions to the controller 900. The input component(s) 906 may include knobs (e.g., as shown in FIGS. 9A-9C), dials, buttons, levers, and/or other actuation mechanisms. An operator can interact with the input component(s) 906 to alter the airflow provided to the two or more pressure-mitigation devices, discharge air from the pressure-mitigation device, or disconnect the controller 900 from the two or more pressure-mitigation devices (e.g., by disconnecting the controller 900 from tubing connected between the controller 900 and the two or more pressure-mitigation devices).

As further discussed below, the controller 900 can be configured to independently inflate and/or deflate one or more chambers of pressure-mitigation devices in a predetermined pattern specific for each pressure-mitigation device by managing one or more flows of fluid (e.g., air) produced by one or more pumps. In some embodiments the pump(s) reside in the housing 902 of the controller 900, while in other embodiments the controller 900 is fluidically connected to the pump(s). For example, the housing 902 may include a first fluid interface through which fluid is received from the pump(s) and a second fluid interface through which fluid is directed to the pressure-mitigation devices. Multi-channel tubing may be connected to either of these fluid interfaces. For example, multi-channel tubing may be connected between the first fluid interface of the controller 900 and multiple pumps. As another example, multi-channel tubing may be connected between the second fluid interface of the controller 900 and multiple valves of the pressure-mitigation devices. Here, the controller 900 includes fluid interfaces 908 designed to interface with multi-channel tubing. In some embodiments the multi-channel tubing permits unidirectional fluid flow, while in other embodiments the multi-channel tubing permits bidirectional fluid flow. Thus, fluid returning from the pressure-mitigation devices (e.g., as part of a discharge process) may travel back to the controller 900 through the second fluid interface. By controlling the exhaust of fluid returning from the pressure-mitigation devices, the controller 900 can actively manage the noise created during use.

By monitoring the connections with the fluid interfaces 908, the controller 900 may be able to detect which type of pressure-mitigation devices have been connected. Each type of pressure-mitigation device may include a different type of connector. For example, a pressure-mitigation device designed for elongated objects (e.g., the pressure-mitigation device 100 of FIGS. 1A-B) may include a first arrangement of magnets in its connector, while a pressure-mitigation device designed for non-elongated objects may include a second arrangement of magnets in its connector. The controller 900 may include one or more sensors arranged near the fluid interfaces 908 that are able to detect whether magnets are located within a specified proximity. The controller 900 may automatically determine, based on which magnets have been detected by the sensor(s), which types of pressure-mitigation devices are connected.

Pressure-mitigation devices may have different geometries, layouts, and/or dimensions suitable for various positions (e.g., supine, prone, sitting), various supporting objects (e.g., wheelchair, bed, recliner, surgical table), and/or various user characteristics (e.g., weight, size, ailment), and the controller 900 can be configured to automatically detect the types of pressure-mitigation devices connected thereto. In some embodiments, the automatic detection is performed using other suitable identification mechanisms, such as the controller 900 reading a radio-frequency identification (RFID) tag or barcode on the pressure-mitigation devices. Alternatively, the controller 900 may permit an operator to specify the types of pressure-mitigation devices connected thereto. For example, the operator may be able to select, using an input component (e.g., input component 906), a type of pressure-mitigation device via a display 910. The controller 900 can be configured to dynamically and independently alter the pattern for inflating and/or deflating chambers based on which types of pressure-mitigation devices are connected.

As shown in FIGS. 9A-9B, the controller 900 may include a display 910 for displaying information related to the pressure-mitigation devices, the pattern of inflations/deflations, the user, etc. For example, the display 910 may present an interface that specifies which types of pressure-mitigation devices are connected to the controller 900. As another example, the display 910 may present an interface that specifies the programmable pattern that is presently governing inflation/deflation of the pressure-mitigation devices, as well as the current state within the programmable patterns for each pressure-mitigation device. Other display technologies could also be used to convey information to an operator of the controller 900. In some embodiments, the controller 900 includes a series of lights (e.g., light-emitting diodes) that are representative of different statuses to provide visual alerts to the operator or the user. For example, a status light may provide a green visual indication if the controller 900 is presently providing therapy, a yellow visual indication if the controller 900 has been paused (i.e., is in a pause mode), a red visual indication if the controller 900 has experienced an issue (e.g., noncompliance of patient, patient not detected) or requires maintenance (i.e., is in an alert mode), etc. These visual indications may dim upon the conclusion of a specified period of time or upon determining that the status has changed (e.g., the pause mode is no longer active).

In some embodiments, the controller 900 includes a rapid deflate function that allows an operator to rapidly and independently deflate pressure-mitigation devices. The rapid deflate function may be designed such that the entirety of a pressure-mitigation device is deflated or a portion (e.g., the side supports) of the pressure-mitigation device is deflated. This may be a software-implemented solution that can be activated via the display 910 (e.g., when configured as a touch-enabled interface) and/or input components (e.g., tactile actuators such as buttons, switches, etc.) on the controller 900. This rapid deflation, in particular the deflation of the side supports, is expected to be beneficial to operators when there is a need for quick access to the user, such as to provide cardiopulmonary resuscitation (CPR).

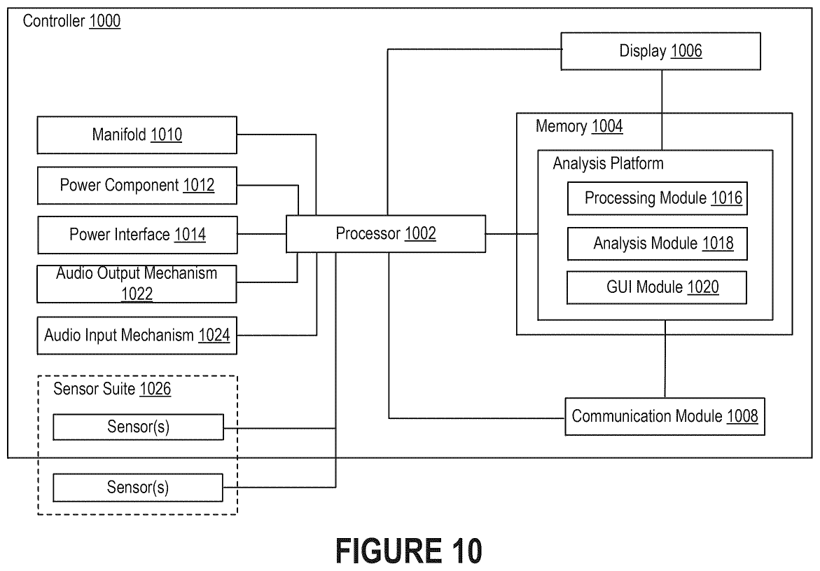

FIG. 10 illustrates an example of a controller 1000 in accordance with embodiments of the present technology. As shown in FIG. 10, the controller 1000 can include a processor 1002, memory 1004, display 1006, communication module 1008, manifold 1010, and/or power component 1012 that is electrically coupled to a power interface 1014. These components may reside within a housing (also referred to as a “structural body”), such as the housing 902 described above with respect to FIGS. 9A-9C. In some embodiments, the aspects of the controller 1000 are incorporated into other components of a pressure-mitigation system. For example, some components of the controller 1000 may be incorporated into a computing device (e.g., a mobile phone or a mobile workstation) that is remotely coupled to two or more pressure-mitigation devices.

Each of these components is discussed in greater detail below. Those skilled in the art will recognize that different combinations of these components may be present depending on the nature of the controller 1000. Other components could also be included depending on the desired capabilities of the controller 1000.

For example, the controller could include one or more fragrance output mechanisms (e.g., spray pumps or spray nozzles) that are able to discharge scented fluid (e.g., air or liquid) from corresponding reservoirs, so as to produce an aroma. Such a feature may be desirable if one of the two or more pressure-mitigation devices is intended to be used as part of a therapy program.

As another example, the controller could include a circuitry that is able to detect and then examine electronic signatures emitted by nearby beacons. Accordingly, if an item (e.g., a wristband or file) that includes a beacon is presented to the controller, the controller may be able to detect the electronic signature emitted by the beacon and then take appropriate action. For instance, the controller may determine, based on the electronic signature that conveys information regarding the human body to be treated, how to independently inflate each of the chambers of the two or more pressure-mitigation devices. Electronic signatures may be transmitted via RFID, Bluetooth, NFC, or another short-range wireless communication protocol. Additionally or alternatively, the controller may be able to examine machine-readable codes (e.g., Quick Response codes, bar codes, and alphanumeric strings) that are printed on items such as wristbands, files, and the like. By examining the machine-readable code that is printed on an item associated with a human body, the controller may be able to determine, infer, or derive information regarding the human body. These features allow a controller to act as a “single action” solution for treating the human body since the controller may automatically begin treatment after an electronic signature or machine-readable code has been presented.

The processor 1002 can have generic characteristics similar to general-purpose processors, or the processor 1002 may be an application-specific integrated circuit (ASIC) that provides control functions to the controller 1000. As shown in FIG. 10, the processor 1002 can be coupled to all components of the controller 1000, either directly or indirectly, for communication purposes.

The memory 1004 may be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 1002, the memory 1004 can also store data generated by the processor 1002 (e.g., when executing the analysis platform). Note that the memory 1004 is merely an abstract representation of a storage environment. The memory 1004 could be comprised of actual memory chips or modules.

The display 1006 can be any mechanism that is operable to visually convey information to an operator. For example, the display 1006 may be a panel that includes LEDs, organic LEDs, liquid crystal elements, or electrophoretic elements. Alternatively, the display 1006 may simply be a series of lights (e.g., LEDs) that are able to indicate the status of the controller 1000. In some embodiments, the display 1006 is touch sensitive. Thus, an operator user may be able to provide input to the controller 1000 by interacting with the display 1006 itself. Additionally or alternatively, the operator may be able to provide input to the controller 1000 by interacting with input components, such as knobs, dials, buttons, levers, and/or other actuation mechanisms.

The communication module 1008 may be responsible for managing communications between the components of the controller 1000, or the communication module 1008 may be responsible for managing communications with other computing devices (e.g., a mobile phone associated with the operator, a network-accessible server system accessible to an entity responsible for manufacturing, providing, or managing pressure-mitigation devices). The communication module 1008 may be wireless communication circuitry that is designed to establish communication channels with other computing devices. Examples of wireless communication circuitry include integrated circuits (also referred to as “chips”) configured for Bluetooth®, Wi-Fi®, Near Field Communication (NFC), and the like.

Moreover, the communication module 1008 may be responsible for providing information for uploading to, and retrieving information from, the electronic health record that is associated with the human body that is presently being treated. Assume, for example, that the controller 1000 receives input indicating that a given person is to be treated using two or more pressure-mitigation devices. In such a situation, the controller 1000 may establish a connection with a storage medium that includes the electronic health record of the given person. In some embodiments the controller 1000 downloads information from the electronic health record into the memory 1004, while in other embodiments the controller 1000 simply accesses the information in the electronic health record. This information could be used to determine how to treat the given person. For example, the controller may determine, based on the weight and age of the given person, which patterns to select for inflating each of the chambers of the two or more pressure-mitigation devices, whether and when to adjust the patterns, etc.

The controller 1000 may be connected to pressure-mitigation devices that each includes a series of chambers whose pressure can be individually varied. When each pressure-mitigation device is placed between a human body and the surface of an object, the controller 1000 can independently cause the pressure on an anatomical region of the human body to be varied by controllably inflating and/or deflating chamber(s). Such action can be accomplished by the manifold 1010, which controls the flow of fluid to the series of chambers of each pressure-mitigation device.

Transducers mounted in the manifold 1010 can generate an electrical signal based on the pressure detected in each chamber of each pressure-mitigation device. Generally, each chamber is associated with a different fluid channel and a different transducer. Accordingly, if the manifold 1010 is designed to facilitate the flow of fluid to a pressure-mitigation device with four chambers, the manifold 1010 may include four fluid channels and four transducers. In some embodiments, the manifold 1010 includes fewer than four fluid channels and/or transducers or more than four fluid channels and/or transducers. Pressure data representative of the values of the electrical signals generated by the transducers can be stored, at least temporarily, in the memory 1004. The manifold 1010 may be driven based on a clock signal that is generated by a clock module (not shown). For example, the processor 1002 may be configured to generate signals for driving valves in the manifold 1010 (or driving integrated circuits in communication with the valves) based on a comparison of the clock signal to programmed patterns that indicate when each chamber of the two or more pressure-mitigation devices should be independently inflated or deflated. The programmed patterns may belong to a set of multiple programmed patterns that are stored in the memory 1004.

An analysis platform may be responsible for examining the pressure data. For convenience, the analysis platform is described as a computer program that resides in the memory 1004. However, the analysis platform could be comprised of software, firmware, or hardware that is implemented in, or accessible to, the controller 1000. In accordance with embodiments described herein, the analysis platform may include a processing module 1016, analysis module 1018, and graphical user interface (GUI) module 1020. Each of these modules can be an integral part of the analysis platform. Alternatively, these modules can be logically separate from the analysis platform but operate “alongside” it. Together, these modules enable the analysis platform to gain insights not only into whether the pressure-mitigation device connected to the controller 1000 is being used properly, but also into the health of the human body situation on or in the two or more pressure-mitigation devices.

The processing module 1016 can process pressure data obtained by the analysis platform into a format that is suitable for the other modules. For example, in preparation for analysis by the analysis module 1018, the processing module 1016 may apply algorithms designed for temporal aligning, artifact removal, and the like. Accordingly, the processing module 1016 may be responsible for ensuring that the pressure data is accessible to the other modules of the analysis platform. As further discussed below, the processor 1002 may forward at least some of the pressure data, in either its processed or unprocessed form, to the communication module 1008 for transmittal to a destination for analysis. In such a scenario, the processing module 1016 may apply operations (e.g., filtering, compressing, labelling) to the pressure data before it is forwarded to the communication module 1008 for transmission to the destination.

By examining the pressure data in conjunction with flow data representative of the fluid flowing into the controller 1000 from the pump(s), the analysis module 1018 can control how the chambers of the pressure-mitigation device are inflated and/or deflated. For example, the analysis module 1018 may be responsible for separately and independently controlling the set point for fluid flowing into each chamber such that the pressures of the chambers match a predetermined pattern for each pressure-mitigation device.

By examining the pressure data, the analysis module 1018 may also be able to sense movements of the human body under which each pressure-mitigation device is positioned. These movements may be caused by the user, another individual (e.g., a caregiver or an operator of the controller 1000), or the underlying surface. The analysis module 1018 may apply algorithms to the data representative of these movements (also referred to as “movement data” or “motion data”) to identify repetitive movements and/or random movements to better understand the health state of the user. For example, the analysis module 1018 may be able to produce a coverage metric indicative of the amount of time that the human body is properly positioned on or in each pressure-mitigation device. As further discussed below, the controller 1000 (or another computing device) may be able to independently establish whether each pressure-mitigation device has been properly deployed and/or operated based on the coverage metric. As another example, the analysis module 1018 may be able to establish the respiration rate, heart rate, or another vital measurement based on the movements of the user. Generally, the movement data are derived from the pressure data. That is, the analysis module 1018 may be able to infer movements of the human body by analyzing the pressure of the chambers of each of the pressure-mitigation devices in conjunction with the rate at which fluid is being delivered to those chambers. Consequently, some embodiments of each of the pressure-mitigation devices may not actually include any sensors for measuring movement, such as accelerometers, tilt sensors, or gyroscopes.

The analysis module 1018 may respond in several ways after examining the pressure data. For example, the analysis module 1018 may generate a notification (e.g., an alert) to be transmitted to another computing device by the communication module 1008. The other computing device may be associated with a medical professional (e.g., a physician or a nurse), a caregiver (e.g., a family member or friend of the user), or some other entity (e.g., a researcher or an insurer). As another example, the analysis module 1018 may cause the pressure data (or analyses of such data) to be integrated with the electronic health record of the user. Generally, the electronic health record is maintained in a storage medium that is accessible to the communication module 1008 across a network.

The GUI module 1020 may be responsible for generating interfaces that can be presented on the display 1006. Various types of information can be presented on these interfaces. For example, information that is calculated, derived, or otherwise obtained by the analysis module 1018 may be presented on an interface for display to the user or operator. As another example, visual feedback may be presented on an interface so as to indicate whether the user is properly situated on or in each pressure-mitigation device.

The controller 1000 may include a power component 1012 that is able to provide to the other components residing within the housing, as necessary. Examples of power components include rechargeable lithium-ion (Li-Ion) batteries, rechargeable nickel-metal hydride (NiMH) batteries, rechargeable nickel-cadmium (NiCad) batteries, etc. In some embodiments, the controller 1000 does not include a power component, and thus must receive power from an external source. In such embodiments, a cable designed to facilitate the transmission of power (e.g., via a physical connection of electrical contacts) may be connected between the power interface 1014 of the controller 1000 and the external source. The external source may be, for example, an alternating current (AC) power socket or another computing device. The cable connected to the power interface 1014 of the controller 1000 may also be able to convey power so as to recharge the power component 1012.

Embodiments of the controller 1000 can include any subset of the components shown in FIG. 10, as well as additional components not illustrated here.

For example, while the controller 1000 is able to receive and transmit data wirelessly via the communication module 1008, other embodiments of the controller 1000 may include a physical data interface through which data can be transmitted to another computing device. Examples of physical data interfaces include Ethernet ports, Universal Serial Bus (USB) ports, and proprietary ports.

As another example, some embodiments of the controller 1000 include an audio output mechanism 1022 and/or an audio input mechanism 1024. The audio output mechanism 1022 may be any apparatus that is able to convert electrical impulses into sound. One example of an audio output mechanism is a loudspeaker (or simply “speaker”). Meanwhile, the audio input mechanism 1024 may be any apparatus that is able to convert sound into electrical impulses. One example of an audio input mechanism is a microphone. Together, the audio output and input mechanisms 1022, 1024 may enable the user or operator to engage in an audible exchange with a person who is not located proximate the controller 1000. Assume, for example, that the user has become misaligned with one or more of the two or more pressure-mitigation devices. In such a scenario, the user may utilize the audio input mechanism 1024 to verbally ask for assistance, for example, from another person who is able to verbally confirm that assistance is forthcoming using the audio output mechanism 1022. The other person could be a medical professional or caretaker of the user. This may be useful in situations where the user is unable to reposition herself on or in one of the pressure-mitigation devices due to an underlying condition that inhibits or prevents movement.

The audio input mechanism 1024 may also be able to generate a signal that is indicative of more nuanced sounds. For example, the audio input mechanism 1024 may generate data that is representative of sounds originating from within the human body situated on or in one or more of the two or more pressure-mitigation devices. These sounds may be representative of auscultation sounds generated by the circulatory, respiratory, and gastrointestinal systems. These data could be transmitted (e.g., by the communication module 1008) to a destination for analysis.

Other sensors may also be implemented in, or accessible to, the controller 1000. For example, sensors may be contained in the housing of the controller 1000 and/or embedded within each pressure-mitigation device that is connected to the controller 1000. Collectively, these sensors may be referred to as the “sensor suite” 1026. For example, the sensor suite 1026 may include a motion sensor whose output is indicative of motion of the controller 1000 or each pressure-mitigation device. Examples of motion sensors include multi-axis accelerometers and gyroscopes. As another example, the sensor suite 1026 may include a proximity sensor whose output is indicative of proximity to the controller 1000 or pressure-mitigation device. A proximity sensor may include, for example, an emitter that is able to emit infrared (IR) light and a detector that is able to detect reflected IR light that is returned toward the proximity sensor. These types of proximity sensors are sometimes called laser imaging, detection, and ranging (LiDAR) scanners. Other examples of sensors include an ambient light sensor whose output is indicative of the amount of light in the ambient environment, a temperature sensor whose output is indicative of the temperature of the ambient environment, and a humidity sensor whose output is indicative of the humidity of the ambient environment. The output(s) produced by the sensor suite 1026 may provide greater insight into the environment in which the controller 1000 is deployed (and thus the environment in which the human body situated on or in each of the two or more pressure-mitigation devices is to be treated).

In some embodiments, the sensor suite 1026 includes one or more specialty sensors that are designed to generate, obtain, or otherwise produce information related to the health of the human body. For example, the sensor suite 1026 may include a vascular scanner. The term “vascular scanner” may be used to refer to an imaging instrument that includes (i) an emitter operable to emit electromagnetic radiation (e.g., in the near infrared range) into the body and (ii) a sensor operable to sense electromagnetic radiation reflected by physiological structures inside the human body. Normally, an image is created based on the reflected electromagnetic radiation that serves as a reference template for the vasculature of an anatomical region. Thus, the vasculature in an anatomical region could be periodically or continually monitored based on outputs produced by a vascular scanner included in the sensor suite 1026. Additionally or alternatively, the sensor suite 1026 may include sensors that are designed to perform pulse oximetry by determining oxygen level of the blood, measure blood pressure, compute heartrate, etc.

Based on the output(s) produced by the sensor suite 1026, the controller 1000 (or some other computing device) may be able to compute some or all of the main vital signs, namely, body temperature, blood pressure, pulse rate, and breathing rate (also referred to as “respiratory rate”). Moreover, the controller 1000 (or some other computing device) may be able to compute metrics that are indicative of the health of the human body, despite not being one of the main vital signs. For example, output(s) generated by the sensor suite 1026 could be used to establish whether the human body is performing a given activity (e.g., sleeping or eating). The output(s) could be used to not only ascertain the sleep pattern of the human body, but also whether changes in the sleep pattern indicate whether the health state of the human body has improved (e.g., sleep more consistent with longer duration following deployment of each pressure-mitigation device).

Note that the sensors included in the sensor suite 1026 need not necessarily be included in the controller 1000. For example, the controller 1000 may be communicatively connected to ancillary sensors that are included in various items (e.g., blankets and clothing), attached to the human body, etc.

These various components may allow the controller 1000 to be readily integrated into a network-connected environment, such as a home or hospital. Thus, the controller 1000 may be communicatively coupled to mobile phones, tablet computers, wearable electronic devices (e.g., fitness trackers and watches), or network-connected devices (also referred to as “smart devices”), such as televisions and home assistant devices. Similarly, the controller 1000 may be communicatively coupled to medical devices, such as cardiac pacemakers, insulin pumps, glucose monitoring devices, and the like. This level of integration can provide several notable benefits over conventional technologies for mitigating pressure.

As an example, the pressure-mitigation system of which the controller 1000 is a part may be used to monitor health of a human body in a more holistic sense. As mentioned above, insights into movements of the human body can be surfaced through analysis of pressure data generated by the controller 1000 or pressure-mitigation devices. Analysis of these movements over an extended period of time (e.g., days, weeks, or months) may lead to the discovery of abnormalities that might otherwise go unnoticed. For example, the controller 1000 (or some other computing device) may infer that the human body is suffering from an ailment in response to a determination that its movements over a recent interval of time differ from those that would be expected based on past intervals of time. At a high level, insights gained through analysis of the pressure data can be used not only to define a “health baseline” for the human body, but also to discover when deviations from the health baseline occur.

As another example, the controller 1000 may be responsible for providing or supplementing prompts to administer medication in accordance with a regimen. Assume, for example, that a user positioned on or in one or more of the two or more pressure-mitigation devices is associated with a regimen that requires a medication be administered regularly. The controller 1000 may promote adherence to the regimen by prompting the user or another person (e.g., an operator of the controller 1000) to administer the medication. Visual notifications could be presented by the display 1006, or audible notifications could be presented by the audio output mechanism 1022. Additionally or alternatively, the controller 1000 could cause digital notifications (also referred to as “electronic notifications”) to be presented by a computing device that is communicatively coupled to the controller 1000. In some embodiments, the regimen is stored in the memory 1004 of the controller 1000. In other embodiments, the regimen is stored in the memory of a computing device that is communicatively coupled to the controller 1000. For example, the regimen may be implemented by a computer program that is executing on a mobile device associated with the user, and when the computer program determines that a dose of the medication is due to be administered, the computer program may transmit an instruction to the controller 1000 to generate a notification.

As another example, the controller 1000 may be able to facilitate communication with medical professionals. Assume, for example, that the controller 1000 is deployed in a home environment that medical professionals visit infrequently or not at all. In such a scenario, the controller 1000 may allow the user to communicate with medical professionals who are located outside of the home environment. Thus, the user may be able to communicate, via the audio output and input mechanisms 1022, 1024, with medical professionals who are located in a hospital environment (e.g., at which the user received treatment) or their own home environments.

As another example, the controller 1000 may be able to facilitate communication with emergency services. For instance, if the controller 1000 determines (e.g., through analysis of pressure data) that no movement has occurred for a predetermined amount of time, the controller 1000 may prompt the user to respond. Similarly, if the controller 1000 receives input from the user indicative of a request for assistance, the controller 1000 may initiate communication with emergency services. Thus, the controller 1000 may be programmed to person some action if, for example, it determines (e.g., through analysis of the signal generated by the audio input mechanism 1024) that the user has indicated she has fallen or has experienced a medical event (e.g., shortness of breath, heart palpitations, excessing sweating).

These benefits allow pressure-mitigation systems to be deployed in situations where frequent visits by medical professionals may not be practical or possible. For example, when deployed in a hospital environment, a pressure-mitigation system may allow medical professionals to visit patients less frequently. Patients situated on or in two or more pressure-mitigation devices may not need to be turned to alleviate pressure as often, and medical professionals may not need to continually check on patients if pressure-mitigation systems are able to autonomously discover changes in health. As another example, when deployed in a home environment, a pressure-mitigation system may be able to counter a lack of visits from medical professionals. If a patient is instructed to situate herself on or in one or more of two or more pressure-mitigation devices while at home, the patient may only need to be visited every few (e.g., three, five, or seven) days rather than once per day or multiple times per day. Overall, implementing pressure-mitigation systems can lead to significant cost savings because medical professionals are required to make less frequent visits and perform fewer medical procedures, and because patients can be discharged more quickly.

The controller 1000 may also be designed to focus on wellness in addition to, or instead of, treatment for (and prevention of) pressure-induced injuries. As an example, embodiments of the controller 1000 may be designed to aid in sleep management, for healthy individuals and/or unhealthy individuals. Using the audio output mechanism 1022 in combination with the manifold 1010, the controller 1000 may be able to accomplish tasks such as simulating the presence of another person, for example, by producing vocal sounds, breathing sounds, applying pressure, and the like.

### Processing System

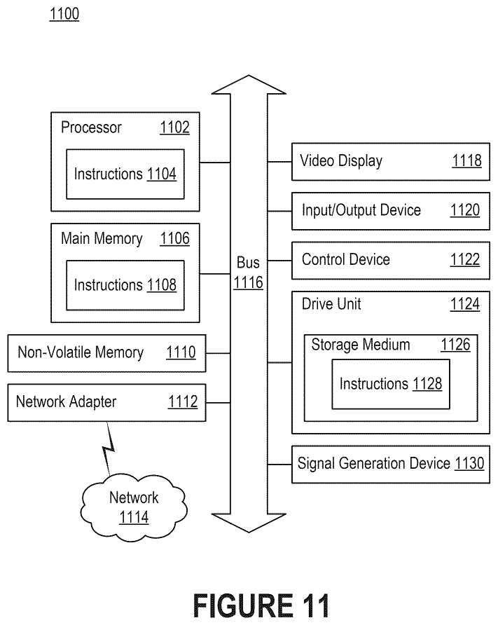

FIG. 11 is a block diagram illustrating an example of a processing system 1100 in which at least some operations described herein can be implemented. For example, components of the processing system 1100 may be hosted on a controller responsible for controlling the flow of fluid to each pressure-mitigation device. As another example, components of the processing system 1100 may be hosted on a computing device that is communicatively coupled to the controller.

The processing system 1100 may include a processor 1102, main memory 1106, non-volatile memory 1110, network adapter 1112 (e.g., a network interface), video display 1118, input/output device 1120, control device 1122 (e.g., a keyboard, pointing device, or mechanical input such as a button), drive unit 1124 that includes a storage medium 1126, or signal generation device 1130 that are communicatively connected to a bus 1116. The bus 1116 is illustrated as an abstraction that represents one or more physical buses and/or point-to-point connections that are connected by appropriate bridges, adapters, or controllers. The bus 1116, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or bus compliant with Institute of Electrical and Electronics Engineers (IEEE) Standard 1194.

The processing system 1100 may share a similar computer processor architecture as that of a computer server, router, desktop computer, tablet computer, mobile phone, video game console, wearable electronic device (e.g., a watch or fitness tracker), network-connected (“smart”) device (e.g., a television or home assistant device), augmented or virtual reality system (e.g., a head-mounted display), or another computing device capable of executing a set of instructions (sequential or otherwise) that specify action(s) to be taken by the processing system 1100.

While the main memory 1106, non-volatile memory 1110, and storage medium 1126 are shown to be a single medium, the terms “storage medium” and “machine-readable medium” should be taken to include a single medium or multiple media that stores one or more sets of instructions 1128. The terms “storage medium” and “machine-readable medium” should also be taken to include any medium that is capable of storing, encoding, or carrying a set of instructions for execution by the processing system 1100.

In general, the routines executed to implement the embodiments of the present disclosure may be implemented as part of an operating system or a specific application, component, program, object, module, or sequence of instructions (collectively referred to as “computer programs”). The computer programs typically comprise one or more instructions (e.g., instructions 1104, 1108, 1128) set at various times in various memories and storage devices in a computing device. When read and executed by the processor 1102, the instructions cause the processing system 1100 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 1110, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 1112 enables the processing system 1100 to mediate data in a network 1114 with an entity that is external to the processing system 1100 through any communication protocol supported by the processing system 1100 and the external entity. The network adapter 1112 can include a network adaptor card, a wireless network interface card, a switch, a protocol converter, a gateway, a bridge, a hub, a receiver, a repeater, or a transceiver that includes an integrated circuit (e.g., enabling communication over Bluetooth or Wi-Fi).

The techniques introduced here can be implemented using software, firmware, hardware, or a combination of such forms. For example, aspects of the present disclosure may be implemented using special-purpose hardwired (i.e., non-programmable) circuitry in the form of application-specific integrated circuits (ASICs), programmable logic devices (PLDs), field-programmable gate arrays (FPGAs), and the like.

### Remarks

The foregoing description of various embodiments has been provided for the purposes of illustration and description. It is not intended to be exhaustive or to limit the disclosed subject matter to the precise forms disclosed. Many modifications and variations will be apparent to one skilled in the art. Embodiments were chosen and described in order to best describe the principles of the invention and its practical applications, thereby enabling those skilled in the relevant art to understand the disclosed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the patent document has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A pressure-mitigation system for dynamically alleviating pressure exerted on a human body at rest, the pressure-mitigation system comprising:
a mattress having an upper surface capable of supporting at least a portion of the human body;
a flexible mattress cover that includes:
an upper cover portion configured to cover the upper surface of the mattress so as to be disposed between the upper surface and the portion of the human body supported by the upper surface,
wherein the upper cover portion includes one or more inflatable chambers that are controllably inflated and deflated to differentially spread pressure exerted upon the portion of the human body,
side cover portions that extend from the upper cover portion and out of a plane of the upper cover portion, and
a cavity defined by the side cover portions and the upper cover portion,
wherein the cavity is configured to fit the mattress therewithin such that the side cover portions extend down a plurality of side surfaces of the mattress; and
a fastening mechanism configured to secure the mattress within the cavity of the flexible mattress cover.

2. The pressure-mitigation system of claim 1, further comprising tubing that extends within the side cover portions of the flexible mattress cover to the one or more inflatable chambers of the upper cover portion, wherein the tubing is configured to transport fluid into or out of the one or more inflatable chambers to inflate or deflate the one or more inflatable chambers.

3. The pressure-mitigation system of claim 1, wherein the one or more inflatable chambers span a planar area that is less than an area of the upper surface of the mattress.

4. The pressure-mitigation system of claim 1, wherein the fastening mechanism is configured to engage with a feature of the mattress so as to secure the flexible mattress cover to the mattress.

5. The pressure-mitigation system of claim 1, wherein the fastening mechanism includes an elastic band that spans through the side cover portions to constrict the side cover portions to the plurality of side surfaces of the mattress.

6. The pressure-mitigation system of claim 1, further comprising a controller located on a side cover portion, wherein the controller includes a processor that executes instructions to inflate and deflate the one or more inflatable chambers.

7. The pressure-mitigation system of claim 1, wherein the flexible mattress cover includes a pocket configured to hold a controller that executes instructions to cause the one or more inflatable chambers to be inflated and deflated.

8. The pressure-mitigation system of claim 1, wherein the one or more inflatable chambers are formed by interconnections between an upper layer and a lower layer of the upper cover portion.

9. The pressure-mitigation system of claim 8, wherein the lower layer interfaces with the upper surface of the mattress, and wherein the fastening mechanism includes non-slip material being included in the lower layer.

10. The pressure-mitigation system of claim 1, wherein the flexible mattress cover is substantially formed of fabric material.

11. A pressure-mitigation apparatus comprising:
a flexible cover having a pressure-mitigation panel region, the flexible cover being configured to lay over a substrate such that the pressure-mitigation panel region is disposed atop an upper surface of the substrate,
wherein the pressure-mitigation panel region includes one or more inflatable chambers that are controllably inflated or deflated,
wherein at least one other region of the flexible cover extends along a different surface of the substrate while the flexible cover is laid over the substrate; and
at least one fastener configured to secure the flexible cover to the substrate while the flexible panel is laid over the substrate.

12. The pressure-mitigation apparatus of claim 11, wherein the at least one fastener is located on the at least one other region of the flexible cover.

13. The pressure-mitigation apparatus of claim 11, wherein the at least one other region includes a plurality of side regions that extend along a plurality of sidewalls of the substrate, and wherein the at least one fastener includes an elastic band that spans through each of the plurality of side regions and that constricts the plurality of side regions around the plurality of sidewalls of the substrate.

14. The pressure-mitigation apparatus of claim 11, wherein the flexible cover includes a pocket configured to fit a controller that causes inflation or deflation of the one or more inflatable chambers.

15. The pressure-mitigation apparatus of claim 11, wherein the at least one fastener is configured to interface with a feature of the substrate.

16. A pressure-mitigation pad comprising:
one or more pad foundations capable of supporting a weight of at least a portion of a human body; and
a pressure-mitigation layer atop the one or more pad foundations to form the pressure-mitigation pad, the pressure-mitigation layer comprising one or more chambers defined by bulkheads within the pressure-mitigation layer,
wherein the one or more chambers are each configured to be inflated or deflated to dynamically control a height of each inflatable chamber relative to the pressure-mitigation layer.

17. The pressure-mitigation pad of claim 16, wherein the one or more pad foundations include tubing that feed into a bottom of the one or more chambers, wherein the tubing transports fluid in or out of the one or more chambers to inflate or deflate the one or more chambers.

18. The pressure-mitigation pad of claim 16, wherein the one or more pad foundations include at least one of foam material, spring coils, or cushioning.

19. The pressure-mitigation pad of claim 16, further comprising a tubing layer through which tubing for the inflatable chambers extends, wherein a side of the tubing layer includes an attachment port that is fluidly connected to the tubing.

20. The pressure-mitigation pad of claim 16, further comprising one or more handles that facilitate transport of the pressure-mitigation pad without separation of the one or more pad foundations and the pressure-mitigation layer.
